(19) United States
(12) Patent Application Publication
(10) Pub. No.: US 2026/0258490 A1
(43) Pub. Date: Sep. 3, 2026

(54) METHODS AND COMPOSITIONS FOR PROBE RESCUE

(71) Applicant: 10x Genomics, Inc., Pleasanton, CA (US)

(72) Inventors: Layla KATIRAEE, Castro Valley, CA (US); Su WANG, Berkeley, CA (US)

(21) Appl. No.: 19/554,465

(22) Filed: Mar. 2, 2026

Related U.S. Application Data
(60) Provisional application No. 63/765,945, filed on Mar. 3, 2025.

(51) Int. Cl. C12Q 1/6869; C12Q 1/6806; C12Q 1/6841; C12Q 1/6855; G01N 21/64

(52) U.S. Cl. CPC C12Q 1/6869; C12Q 1/6806; C12Q 1/6841; C12Q 1/6855; G01N 21/6458

(57) ABSTRACT

The present disclosure relates in some aspects to methods for probing a target nucleic acid in a biological sample (e.g., cell or tissue sample) using a plurality of circularizable probes. Provided herein are methods and compositions for performing an assay using a plurality of circularizable probes that are gap filled and ligated.

### CROSS-REFERENCE TO RELATED APPLICATIONS

This application claims priority to U.S. Provisional Patent Application No. 63/765,945, filed Mar. 3, 2025, entitled “METHODS AND COMPOSITIONS FOR PROBE RESCUE,” which is herein incorporated by reference in its entirety for all purposes.

### FIELD

The present disclosure relates in some aspects to methods and compositions for processing nucleic acid molecules for probing a target nucleic acid in a biological sample (e.g., cell or tissue sample) using a plurality of circularizable probes, such as for detection of a nucleic acid sequence in situ in a biological sample, e.g., in a cell or tissue sample.

### BACKGROUND

Methods are available for analyzing nucleic acids present in a biological sample, such as a cell or a tissue. However, probe-based multiplexed analysis of a panel of transcripts has remained challenging and assays may require costly reagents. Improved methods for analyzing nucleic acids present in a biological sample are needed. Provided herein are methods and compositions that address such and other needs.

### BRIEF SUMMARY

In some aspects, provided herein is a method comprising: contacting a biological sample with a plurality of circularizable probes comprising a full-length circularizable probe and a less-than-full-length circularizable probe, wherein the plurality of circularizable probes target the same target region of a target nucleic acid, and the circularizable probes of the plurality of circularizable probes comprise a first hybridization sequence at the 5′ end and a second hybridization sequence at the 3′ end, wherein the first hybridization sequence and the second hybridization sequence hybridize to a sequence of at least a portion of the target region, and wherein the 5′ terminal nucleotide and the 3′ terminal nucleotide of the full-length circularizable probe hybridize to adjacent nucleotides in the target region; filling a gap between the first hybridization sequence and the second hybridization sequence of the less-than-full-length circularizable probe to form a gap-filled circularizable probe; and forming a plurality of circularized probes from the full-length circularizable probe and the gap-filled circularizable probe in the biological sample.

In some embodiments, the less-than-full-length circularizable probe is at least one nucleotide shorter than the full-length circularizable probe. In some embodiments, the less-than-full-length circularizable probe is more than two nucleotides shorter than the full-length circularizable probe.

In some embodiments, at least 20% of probes in the plurality of circularizable probes are less-than-full-length circularizable probes.

In some embodiments, the less-than-full-length circularizable probe is missing a nucleotide at the 5′ end of the first hybridization sequence corresponding to the nucleotide at the 5′ terminus of the full-length circularizable probe.

In some embodiments, the plurality of circularizable probes are synthesized from 3′ to 5′. In some embodiments, synthesis of the plurality of circularizable probes comprises coupling of at least 50 nucleotides, at least 60 nucleotides, at least 70 nucleotides, at least 80 nucleotides, or at least 90 nucleotides.

In some embodiments, the method comprises contacting the biological sample with a ligase during or prior to filling the gap between the first hybridization sequence and the second hybridization sequence. In some embodiments, the full-length circularizable probes are ligated prior to filling the gap between the first hybridization sequence and the second hybridization sequence of the less-than-full-length circularizable probe. In some embodiments, the method further comprises contacting the biological sample with an additional ligase after filling the gap between the first hybridization sequence and the second hybridization sequence to form the plurality of circularized probes. In some embodiments, the method comprises contacting the biological sample with a first ligase during or prior to filling the gap between the first hybridization sequence and the second hybridization sequence and contacting the biological sample with an additional ligase after filling the gap between the first hybridization sequence and the second hybridization sequence to form the plurality of circularized probes.

In some embodiments, the method comprises contacting the biological sample with a ligase after filling the gap between the first hybridization sequence and the second hybridization sequence.

In some embodiments, the ligase is a T4 DNA ligase or a T4 RNA ligase.

In some embodiments, the gap is filled using a DNA polymerase. In some embodiments, the DNA polymerase is selected from the group consisting of Sulfolobus DNA Polymerase I, Klenow fragment, and Hemo KlenTaq® DNA polymerase.

In some embodiments, the biological sample is contacted with a plurality of dNTPs for filling the gap between the first hybridization sequence and the second hybridization sequence.

In some embodiments, forming the plurality of circularized probes comprises performing an RNA templated ligation.

In some embodiments, forming the plurality of circularized probes comprises performing a DNA templated ligation.

In some embodiments, the less-than-full-length circularizable probe comprises a non-phosphorylated 5′ end. In some embodiments, the method further comprises using a kinase to phosphorylate the non-phosphorylated 5′ end of the less-than-full-length circularizable probe. In some embodiments, the phosphorylation of the non-phosphorylated 5′ end of the less-than-full-length circularizable probe is performed in a buffer comprising ATP. In some embodiments, the phosphorylation is performed in vitro prior to contacting the biological sample with the plurality of circularizable probes. In some embodiments, the phosphorylation is performed in the biological sample after contacting the biological sample with the plurality of circularizable probes. In some embodiments, the phosphorylation is performed after filling the gap. In some embodiments, the phosphorylation of the non-phosphorylated 5′ end of the less-than-full-length circularizable probe is performed in a ligation buffer. In some embodiments, the plurality of circularizable probes is incubated with the kinase at about 30° C. to about 40° C. In some embodiments, the plurality of circularizable probes is incubated with the kinase for at least 15 minutes, at least 30 minutes, at least 1 hour or at least 2 hours. In some embodiments, the kinase is a T7 or T4 Polynucleotide Kinase (PNK).

In some embodiments, forming the plurality of circularized probes comprises enzymatic ligation.

In some embodiments, forming the plurality of circularized probes comprises chemical ligation.

In some embodiments, the method further comprises generating a plurality of amplification products using the plurality of circularized probes as templates. In some embodiments, the plurality of amplification products comprises a plurality of rolling circle amplification (RCA) products.

In some embodiments, circularized probes of the plurality of circularized probes comprise one or more barcode sequences. In some embodiments, the one or more barcode sequences are associated with the target nucleic acid. In some embodiments, the plurality of amplification products comprises the one or more barcode sequences or complementary sequences thereof.

In some embodiments, the method further comprises detecting the one or more barcode sequences or complementary sequences thereof in the biological sample. In some embodiments, the plurality of amplification products each comprises multiple copies of each of the one or more barcode sequences or complements thereof.

In some embodiments, detecting the one or more barcode sequences or complement thereof comprises: contacting the biological sample with a detectably labeled probe, wherein the detectably labeled probe comprises a hybridization region complementary to one barcode sequence or complement thereof of the one or more barcode sequences or complements thereof, and detecting complexes formed between the one or more barcode sequences or complements thereof and the detectably labeled probe.

In some embodiments, detecting the one or more barcode sequences or complements thereof comprises: contacting the biological sample with a universal pool of detectably labeled probes and a first pool of intermediate probes, wherein an intermediate probe of the first pool of intermediate probes comprise hybridization regions complementary to the one or more barcode sequences or complements thereof and reporter regions complementary to a detectably labeled probe of the universal pool of detectably labeled probes; and detecting complexes formed between the barcode sequences or complements thereof, the intermediate probe of the first pool of intermediate probes, and the detectably labeled probe of the universal pool of detectably labeled probes. In some embodiments, the method further comprises removing the first pool of intermediate probes and the universal pool of detectably labeled probes.

In some embodiments, an individual barcode sequence or complement thereof of the one or more barcode sequences or complements thereof is assigned a series of signal codes that identifies the individual barcode sequence or complement thereof, and wherein detecting the one or more barcode sequences or complements thereof comprises decoding the individual barcode sequence or complement thereof by detecting the corresponding series of signal codes detected from sequential hybridization, detection, and removal of sequential pools of intermediate probes and the universal pool of detectably labeled probes.

In some embodiments, detecting the one or more barcode sequences or complements thereof comprises performing in situ sequencing-by-synthesis (SBS), sequencing-by-avidity (SBA) or sequencing-by-binding (SBB) in the biological sample.

In some embodiments, the detectably labeled probes are fluorescently labeled.

In some embodiments, the plurality of amplification products of the circularized probe is generated and detected at a plurality of locations in the biological sample.

In other aspects, provided herein is a method comprising: contacting a biological sample with a plurality of circularizable probes comprising a full-length circularizable probe and a less-than-full-length circularizable probe, wherein the plurality of circularizable probes target the same target region of a target nucleic acid, and the circularizable probes of the plurality of circularizable probes comprise one or more barcode sequences, a first hybridization sequence at the 5′ end, and a second hybridization sequence at the 3′ end, wherein the first hybridization sequence and the second hybridization sequence hybridize to a sequence of at least a portion of the target region, wherein the full-length and less-than-full-length circularizable probes hybridize to different target nucleic acid molecules comprising the same target region, and wherein the 5′ terminal nucleotide and the 3′ terminal nucleotide of the full-length circularizable probe hybridize to adjacent nucleotides in the target region; using a polymerase to fill a gap between the first hybridization sequence and the second hybridization sequence of the less-than-full-length circularizable probe to form a gap-filled circularizable probe; and forming a plurality of circularized probes from the full-length circularizable probe and the gap-filled circularizable probe at a plurality of locations in the biological sample; generating a plurality of rolling circle amplification (RCA) products using the plurality of circularized probes as templates; and detecting the one or more barcode sequences or complements thereof in the generated RCA products at the plurality of locations in the biological sample.

In some embodiments, the less-than-full-length circularizable probe is at least one nucleotide shorter than the full-length circularizable probe and at least 20% of probes in the plurality of circularizable probes are less-than-full-length circularizable probes.

In some embodiments, the method comprises contacting the biological sample with a ligase after filling the gap between the first hybridization sequence and the second hybridization sequence.

In some embodiments, the method further comprises using a kinase to phosphorylate a non-phosphorylated 5′ end of the less-than-full-length circularizable probe. In some embodiments, the phosphorylation is performed in vitro prior to contacting the biological sample with the plurality of circularizable probes. In some embodiments, the phosphorylation is performed in the biological sample after contacting the biological sample with the plurality of circularizable probes.

In some embodiments, detecting the one or more barcode sequences or complements thereof comprises: contacting the biological sample with a universal pool of detectably labeled probes and a first pool of intermediate probes, wherein an intermediate probe of the first pool of intermediate probes comprise hybridization regions complementary to the one or more barcode sequences or complements thereof and reporter regions complementary to a detectably labeled probe of the universal pool of detectably labeled probes; and detecting complexes formed between the barcode sequences or complements thereof, the intermediate probe of the first pool of intermediate probes, and the detectably labeled probe of the universal pool of detectably labeled probes.

In some embodiments, detecting the one or more barcode sequences or complements thereof comprises performing in situ sequencing-by-synthesis (SBS), sequencing-by-avidity (SBA) or sequencing-by-binding (SBB) in the biological sample.

In some embodiments, the biological sample comprises a plurality of target nucleic acids and the biological sample is contacted with an additional plurality of circularizable probes that target the target region of an additional target nucleic acid of the plurality of target nucleic acids.

In some embodiments, the target nucleic acid is an endogenous cellular target nucleic acid or a product thereof.

In some embodiments, the target nucleic acid is RNA. In some embodiments, the target nucleic acid is an mRNA.

In some embodiments, the target nucleic acid is genomic DNA.

In some embodiments, the target nucleic acid is cDNA.

In some embodiments, the target nucleic acid is immobilized in the biological sample.

In some embodiments, the target nucleic acid is crosslinked to one or more molecules in the biological sample, a matrix such as a hydrogel, and/or one or more functional groups on a substrate.

In some embodiments, the detecting comprises imaging the biological sample to detect signals associated with the target nucleic acid in situ.

In some embodiments, the biological sample is a cell or tissue sample. In some embodiments, the cell or tissue sample is a processed or cleared cell or tissue sample.

In some embodiments, the biological sample is embedded in a hydrogel.

In other aspects, provided herein is a system comprising: a biological sample comprising a first target nucleic acid molecule and a second target nucleic acid molecule, wherein the biological sample is on a solid support; and a plurality of circularizable probes comprising a full-length circularizable probe and a less-than-full-length circularizable probe, wherein: circularizable probes of the plurality of circularizable probes comprises a first hybridization sequence at the 5′ end and a second hybridization sequence at the 3′ end, the full-length circularizable probe and the less-than-full-length circularizable probe comprise phosphorylated 5′ ends, the first and second hybridization sequences of the full-length circularizable probe are complementary to a target region of the first target nucleic acid molecule, and the first and second hybridization sequences of the less-than-full-length circularizable probe are complementary to a target region of the second target nucleic acid molecule, and the target region of the first target nucleic acid molecule and the second target nucleic acid molecule are the same target region, wherein the same target region comprises the same nucleic acid sequence.

In some embodiments, the less-than-full-length circularizable probe is at least one nucleotide shorter than the full-length circularizable probe, wherein the 5′ terminal nucleotide and the 3′ terminal nucleotide of the full-length circularizable probe are configured to hybridize to adjacent nucleotides in the target region.

In some embodiments, the system further comprises a ligase for forming a plurality of circularized probes from the full-length circularizable probe and the less-than-full-length circularizable probe.

In some embodiments, the system further comprises a polymerase, wherein the polymerase is for filling a gap in the less-than-full-length circularizable probe.

In some embodiments, the system further comprises one or more reagents for performing rolling circle amplification (RCA).

In other aspects, provided herein is a kit comprising: a plurality of circularizable probes comprising a full-length circularizable probe and a less-than-full-length circularizable probe, wherein: the plurality of circularizable probes target the same target region of a target nucleic acid, and each circularizable probe of the plurality of circularizable probes comprises a first hybridization sequence at the 5′ end and a second hybridization sequence at the 3′ end, the first hybridization sequence and the second hybridization sequence are complementary to at least a portion of the target region, and the 5′ terminal nucleotide and the 3′ terminal nucleotide of the full-length circularizable probe are complementary to adjacent nucleotides in the target region; a kinase; a ligase; and a polymerase.

In some embodiments, the polymerase is a DNA Polymerase.

In some embodiments, the kit further comprises a plurality of dNTPs.

In some embodiments, the less-than-full-length circularizable probe is at least two nucleotides shorter than the full-length circularizable probe.

In some embodiments, at least one probe of the plurality of circularizable probes comprises a non-phosphorylated 5′ end.

In some embodiments, the kinase is a T7 or T4 Polynucleotide Kinase (PNK).

In some embodiments, at least 20% of probes in the plurality of circularizable probes are less-than-full-length circularizable probes.

In some embodiments, the plurality of circularizable probes are synthesized from 3′ to 5′. In some embodiments, synthesis of the plurality of circularizable probes comprises coupling of at least 50 nucleotides, at least 60 nucleotides, at least 70 nucleotides, at least 80 nucleotides, or at least 90 nucleotides.

In some embodiments, the kit further comprises one or more reagents for performing rolling circle amplification (RCA). In some embodiments, the one or more reagents for performing RCA comprises a Phi29 DNA polymerase.

In some embodiments, the plurality of circularized probes each comprises one or more barcode sequences. In some embodiments, the kit further comprises a universal pool of detectably labeled probes and a pool of intermediate probes, wherein the intermediate probes of the pool of intermediate probes comprise (i) hybridization regions complementary to the one or more barcode sequences or complements thereof and (ii) reporter regions complementary to a detectably labeled probe of the universal pool of detectably labeled probes. In some embodiments, the kit further comprises one or more reagents for performing sequencing-by-ligation, sequencing-by-synthesis, sequencing-by-avidity, sequencing-by-binding, or a combination thereof.

In other aspects, provided herein is a system comprising: a plurality of circularizable probes comprising a full-length circularizable probe and a less-than-full-length circularizable probe, wherein: the plurality of circularizable probes targets the same target region of a target nucleic acid, and circularizable probes of the plurality of circularizable probes comprises a first hybridization sequence at the 5′ end and a second hybridization sequence at the 3′ end, the full-length circularizable probe and the less-than-full-length circularizable probe comprise phosphorylated 5′ ends, and the first hybridization sequence and the second hybridization sequence are complementary to at least a portion of the target region; a biological sample, wherein the biological sample comprises the target nucleic acid, wherein the biological sample is on a solid support; a ligase; and a polymerase for filling a gap in the less-than-full-length circularizable probe.

In some embodiments, the polymerase is a DNA Polymerase.

In some embodiments, the ligase is a T4 DNA ligase or a T4 RNA ligase.

In some embodiments, the system further comprises a plurality of dNTPs for filling a gap in the less-than-full-length circularizable probe.

In some embodiments, the less-than-full-length circularizable probe is at least one nucleotide shorter than the full-length circularizable probe, wherein the 5′ terminal nucleotide and the 3′ terminal nucleotide of the full-length circularizable probe are complementary to adjacent nucleotides in the target region. In some embodiments, the less-than-full-length circularizable probe is more than two nucleotides shorter than the full-length circularizable probe.

In some embodiments, at least 20% of probes in the plurality of circularizable probes are less-than-full-length circularizable probes.

In some embodiments, the plurality of circularizable probes are synthesized from 3′ to 5′. In some embodiments, synthesis of the plurality of circularizable probes comprises coupling of at least 50 nucleotides, at least 60 nucleotides, at least 70 nucleotides, at least 80 nucleotides, or at least 90 nucleotides.

In some embodiments, the system further comprises one or more reagents for performing rolling circle amplification (RCA). In some embodiments, the one or more reagents for performing RCA comprises a Phi29 DNA polymerase.

In some embodiments, the plurality of circularized probes each comprises one or more barcode sequences. In some embodiments, the system further comprises a universal pool of detectably labeled probes and a pool of intermediate probes, wherein the intermediate probes of the pool of intermediate probes comprise (i) hybridization regions complementary to the one or more barcode sequences or complements thereof and (ii) reporter regions complementary to a detectably labeled probe of the universal pool of detectably labeled probes. In some embodiments, the system further comprises one or more reagents for performing sequencing-by-ligation, sequencing-by-synthesis, sequencing-by-avidity, sequencing-by-binding, or a combination thereof.

### BRIEF DESCRIPTION OF THE DRAWINGS

The following drawings illustrate certain embodiments of the features and advantages of this disclosure. These embodiments are not intended to limit the scope of the appended claims in any manner.

FIG. 1 shows the theoretical full-length product (%) of synthesized oligonucleotides of various lengths.

FIG. 2A depicts a plurality of circularizable probes synthesized from the 3′ end comprising less-than-full-length probe oligonucleotides that are short one or more nucleotides compared to full-length probe oligonucleotides that are phosphorylated at the 5′ end. Upon hybridization to a target nucleic acid, the 5′ and 3′ hybridization sequences of the full-length probe (n nucleotides in length) is positioned for ligation (top right) while a gap (x nucleotides) is present between the 5′ and 3′ hybridization sequences of the less-than-full-length probe (bottom right).

FIG. 2B shows a workflow for generating a plurality of circularized probes from circularizable probes comprising a full-length oligonucleotide (Probe 2) and a less-than-full-length oligonucleotide short at least one nucleotide (Probe 1) compared to the full-length oligonucleotide. The synthesized probe oligonucleotides are treated in vitro with a phosphate modification, contacted with the target nucleic acid, gap filled (for Probe 1), and ligated to generate circularized probes for use as a template in a rolling circle amplification (RCA) reaction. In some embodiments, the treatment to phosphorylate the synthesized probe oligonucleotides is performed in situ before the circularized probes are generated by ligation (e.g., between the gap fill and ligation).

FIG. 3 shows the results of decoded transcripts in assays performed with and without gap fill and/or polynucleotide kinase (PNK) treatment in three different lots of synthesized probes.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

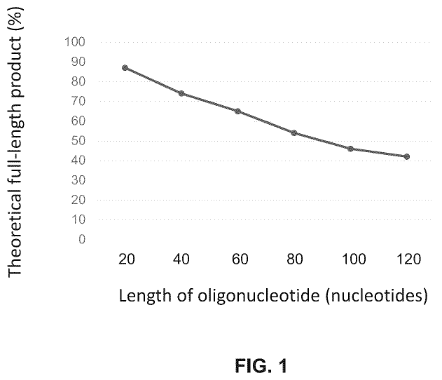

Methods for detecting target nucleic acid use the target nucleic acid to aid the ligation of a probe, such as a circularizable probe (e.g., a padlock probe), where a ligation event occurs if a matching circularizable probe hybridizes to the corresponding target nucleic acid. In some cases, circularizable probes are linear nucleic acid molecules that comprise sequences complementary to the target nucleic acid at their 5′ and 3′ ends. Upon binding (e.g., hybridization) of a circularizable probe to its target nucleic acid, the 5′ and 3′ ends are brought in proximity to enable ligation. Ligation occurs if the 5′ and 3′ ends of the circularizable probe are positioned in proximity for the ligase to join the ends of the probe. Ligation-based assays are useful for detecting a plurality of molecules of the same target nucleic acids in various locations of a biological sample. To detect a plurality of molecules of the same target nucleic acid, multiple probe molecules are provided for binding the same target region. However, providing a plurality of probe molecules requires synthesis of probe oligonucleotides. Synthesis of oligonucleotides is not only costly, it can also vary in quality as oligonucleotide length increases. In some cases, a population of synthesized oligonucleotides may have a significant portion of less-than-full-length products due to errors in synthesis. For example, during synthesis of an oligonucleotide, the 5′ terminal nucleotide is added last during the synthesis cycle, and any incomplete coupling reactions or premature termination results in a truncated, less-than-full-length oligonucleotide lacking the intended terminal 5′ nucleotide or sequence (e.g., lacking one or more intended 5′ terminal nucleotides). As used herein, a “less-than-full-length” circularizable probe is a nucleic acid molecule that is short by at least one nucleotide compared to the full-length product (e.g., short by at least one, at least two, at least three, at least four, or more, nucleotides compared to the full-length product). The less-than-full-length oligonucleotides may also be missing critical modifications such as phosphorylation of the 5′ end which is necessary for ligation of the probe in a detection assay. Synthesis of the plurality of circularizable probes comprises incorporation of nucleotides based on a coupling efficiency. With average methods, the coupling efficiency is about 99.1% to 99.4%. Using the formula: percent full-length product=[(eff)\hat{ }(n−1)]*100 where eff=coupling efficiency (e.g., 0.991-0.994) and (n−1) is the number of coupling reactions needed to make a full-length oligonucleotide of length n. FIG. 1 shows the theoretical yield for different lengths of oligonucleotides of n. As shown in FIG. 1, average oligonucleotide synthesis methods can have a theoretical yield of about 55% full-length product of about 80 nucleotides, and the theoretical yield decreases to about 45% full-length product of about 100 nucleotides. When used in a detection assay, these probe oligonucleotides that are short at least one nucleotide hybridize to the target nucleic acid but cannot be ligated nor amplified and may reduce the overall sensitivity of the assay. In some instances, a less-than-full-length probe has lower affinity for hybridization to the target nucleic acid compared to a full-length probe and/or is more likely to be removed after hybridization during a wash.

The present disclosure provides methods and compositions for using a plurality of circularizable probes to target the same target region of a target nucleic acid. For example, the plurality of circularizable probes comprises a full-length circularizable probe (e.g., a first probe) and a less-than-full-length circularizable probe (e.g., a second probe). In some embodiments, the circularizable probes of the plurality of circularizable probes comprises a hybridization region comprising a first hybridization sequence at the 5′ end and a second hybridization sequence at the 3′ end, wherein the first hybridization sequence and the second hybridization sequence are complementary to at least a portion of the target region, and at least one probe of the plurality of circularizable probes is different in length than other probes in the plurality. For example, the less-than-full-length circularizable probe is short by at least one nucleotide compared to a full-length probe. In some cases, the less-than-full-length circularizable probe (e.g., the second probe) is short by at least two nucleotides compared to a full-length probe (e.g., the first probe). In some cases, at least less-than-full-length circularizable probe is short by at least one nucleotide compared to a full-length probe and comprises a non-phosphorylated 5′ end. After hybridization, a gap between the first hybridization sequence and the second hybridization sequence of a less-than-full-length circularizable probe is filled using the target nucleic acid as template, and a plurality of circularized probes are formed in the biological sample. The methods provided herein may comprise using a polymerase to fill a gap at the 5′ end of a less-than-full-length circularizable probe, thereby forming a functional and ligatable probe.

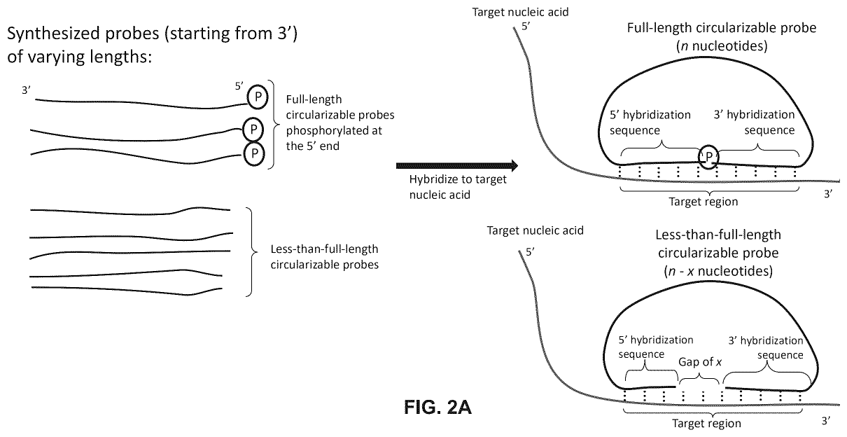

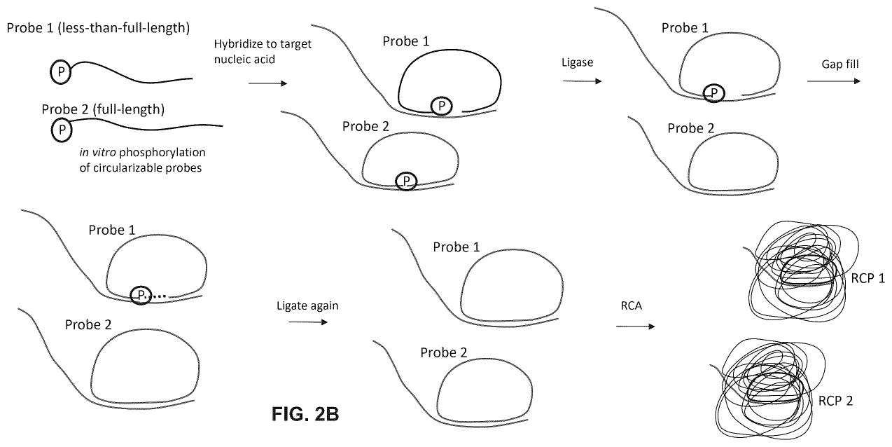

FIG. 2A depicts a plurality of circularizable probes synthesized from the 3′ end comprising full-length probe oligonucleotides and less-than-full-length probe oligonucleotides. In some embodiments, “full-length” circularizable probe refers to an oligonucleotide with a sequence of length n and “less-than-full-length” circularizable probe refers to an oligonucleotide with a length of n−x, wherein n and x are both positive integers. In some instances, the less-than-full-length circularizable probe has a length of n−1, n−2, n−3, n−4, n−5, n−6, n−7, n−8, n−9, or n−10. In some aspects, the full-length circularizable probe has a length of n nucleotides and the less-than-full-length circularizable probe has a length of n−x, wherein x is greater than 2, 3, 4, 5, 6, 7, 8, 9 or 10 or more nucleotides. In some embodiments, the full-length circularizable probe is phosphorylated at the 5′ end. In some embodiments, “full-length” circularizable probe refers to an oligonucleotide with a sequence of length n with a 5′ phosphate modification and “less-than-full-length” circularizable probe refers to an oligonucleotide with a length of n−x without a 5′ phosphate modification, wherein x is greater than or equal to 1. In some instances, a plurality of circularizable probes comprises a plurality of less-than-full-length circularizable probes of various lengths. In some embodiments, upon hybridization to a target nucleic acid, the 5′ and 3′ hybridization sequences of a full-length probe (n nucleotides in length) is positioned for ligation while a gap (x nucleotides) is present between the 5′ and 3′ hybridization sequences of the less-than-full-length circularizable probe. In some embodiments, the sequence of the full-length circularizable probe is designed such that the 5′ terminal nucleotide and the 3′ terminal nucleotide of the full-length circularizable probe hybridize to adjacent nucleotides in the target region of the target nucleic acid. In some instances, a less-than-full-length circularizable probe is short one or more nucleotides (i.e., one or more 5′ terminal nucleotides) from its intended full length. For example, a less-than-full-length circularizable probe may be short one, two, three, four, or more 5′ terminal nucleotides, wherein the less-than-full-length circularizable probe otherwise comprises the same sequence as the corresponding full-length circularizable probe (i.e., except for the one, two, three, four, or more 5′ terminal nucleotides short). In some embodiments, the sequence of the corresponding full-length circularizable probe is designed such that the 5′ terminal nucleotide and the 3′ terminal nucleotide of the full-length circularizable probe hybridize to adjacent nucleotides in the target region of the target nucleic acid. In some embodiments, the adjacent nucleotides in the target region are directly adjacent nucleotides (e.g., do not comprise a gap between each nucleotide of the adjacent nucleotides).

In some embodiments, the less-than-full-length circularizable probe is missing one or more nucleotides of the 5′ end of the full-length circularizable probe. In some embodiments, the less-than-full-length circularizable probe is missing one or more nucleotides of the first hybridization sequence at the 5′ end of the full-length circularizable probe. For example, when the sequence of a full-length circularizable probe and a corresponding less-than-full-length circularizable probe are aligned, one or more nucleotides at the 5′ end of the full-length circularizable probe is missing at the 5′ end of the less-than-full-length circularizable probe. In some instances, a full-length circularizable probe comprises a 20-nucleotiode first hybridization sequence at the 5′ end and a 20-nucleotiode second hybridization sequence at the 3′ end. In some instances, a less-than-full-length circularizable probe targeting the same target region of the target nucleic acid comprises a first hybridization sequence at the 5′ end that is less than 20 nucleotides and the same 20-nucleotiode second hybridization sequence at the 3′ end as the full-length circularizable probe. In some instances, a less-than-full-length circularizable probe comprises a first hybridization sequence at the 5′ end that is 12, 13, 14, 15, 16, 17, 18, or 19 nucleotides and the same 20-nucleotiode second hybridization sequence at the 3′ end compared to the full-length circularizable probe comprising a 20-nucleotiode first hybridization sequence at the 5′ end and a 20-nucleotiode second hybridization sequence at the 3′ end.

In some instances, a plurality of circularizable probes comprises at least 10%, at least 20%, at least 30% or at least 40% of probes in the plurality of circularizable probes are shorter than the full-length probe. In some instances, a plurality of circularizable probes comprises at least 10%, at least 20%, at least 30% or at least 40% less-than-full-length circularizable probes. In some instances, a plurality of circularizable probes designed to have a full-length sequence of at least 90 nucleotides in length comprises at least 20% less-than-full-length circularizable probes. In some instances, a plurality of circularizable probes designed to have a full-length sequence of at least 90 nucleotides in length comprises at least 30% less-than-full-length circularizable probes.

In some embodiments, at least one probe of the plurality of circularizable probes comprises a non-phosphorylated 5′ end and is treated with a kinase to phosphorylate the end of the non-phosphorylated 5′ end of the probe. In some aspects, the method comprises, ligating a plurality of the circularizable probes in the biological sample. In some aspects, compared to using a plurality of circularizable probes with gap filling less-than-full-length circularizable probes, the gap filling increases sensitivity of the assay. The provided methods allow rescue of less-than-full-length circularizable probes that are short by one or more nucleotides compared to a full-length probe by performing gap fill using the target nucleic acid as template after hybridization of the less-than-full-length circularizable probes to the target nucleic acid. In some embodiments, the gap filling is performed as part of the in situ assay. In some embodiments, the detection of a sequence described herein provides information regarding the location and/or identity of an associated target nucleic acid (e.g., hybridized by the plurality of circularizable probe(s)) in a sample.

In some embodiments, a plurality of circularizable probes hybridize to molecules of a target RNA in the biological sample. In some embodiments, the target RNA is a target mRNA. In some embodiments, after kinase treatment of the circularizable probes and gap filling less-than-full-length circularizable probes, an RNA templated DNA ligation is performed. In some embodiments, a DNA templated ligation is performed.

Provided herein are circularizable probes for in situ applications and analyte detection. In some embodiments, only probes that are hybridized to a target nucleic acid have a 5′ phosphate at the end, and are positioned to have the 5′ and 3′ ends of the probe for ligation for use in downstream amplification and/or detection. In some embodiments, a plurality of probes hybridized to different molecules of the same target nucleic acid are processed as described herein.

The present disclosure also provides probes, compositions, kits, systems, and devices for use in accordance with the provided methods. In some aspects, the provided methods and systems can be applied to detect, image, quantitate, or determine the presence or absence of one or more target nucleic acid(s) or portions thereof. In some aspects, the provided methods can be applied to detect, image, quantitate, or determine the sequence of one or more target nucleic acid(s). In some aspects, the provided embodiments are employed for in situ detection and/or sequencing of a target nucleic acid in a cell, e.g., in cells of a biological sample or a sample derived from a biological sample, such as a tissue section on a solid support, such as on a transparent slide. In some aspects, the provided embodiments are used to detect a target nucleic acid or a sequence thereof in a deposited cell sample on a solid support, such as on a transparent slide.

In some aspects, provided herein are in situ assays using microscopy as a readout, e.g., nucleic acid sequencing, hybridization, or other detection or determination methods involving an optical readout. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product) generated using the provided probes (e.g., circularizable probes).

In some embodiments, through the use of various probes and processing workflows (e.g., various circularizable probes such as described in Section II), the present disclosure provides methods for high-throughput profiling a large number of targets in situ, such as transcripts and/or DNA loci, for detecting and/or quantifying nucleic acids in cells, tissues, organs or organisms.

In some embodiments, the provided methods involve analyzing, e.g., detecting or determining, one or more sequences present in a plurality of circularizable probes and/or in a plurality of amplification products generated using the circularized probes. In some embodiments, the plurality of generated amplification products are associated with the same target nucleic acid (e.g., the same mRNA). In some embodiments, the analysis is used to correlate a sequence detected in a plurality of amplification products to a plurality of circularizable probes. In some embodiments, the detection of a sequence in a plurality of amplification products provide information regarding the locations and/or identity of an associated target nucleic acid (e.g., hybridized by the plurality of circularizable probes) in a sample. In some aspects, the provided methods comprise one or more steps of ligating the polynucleotides, for instance of ligating the ends of a circularizable probe (including gap filled less-than-full-length circularizable probes) to form a circularized probe. In some aspects, the provided methods comprise amplifying a plurality of circularized probes to generate a plurality of amplification products. In some aspects, the provided methods involve a step of detecting and/or determining the sequence of all or a portion of the plurality of amplification products.

In some aspects, the provided methods can be applied for various applications, such as for in situ analysis, comprising in situ detection (e.g., based on hybridization such as sequential hybridization) and/or sequencing of target nucleic acids and multiplexed nucleic acid analysis. In some aspects, the provided methods are employed for in situ analysis of target nucleic acids, for example for in situ sequencing or multiplexed analysis in intact tissues or a sample with preserved cellular or tissue structure.

In some embodiments, provided herein are methods for assessing one or more target nucleic acids, such as a plurality of mRNAs, in a biological sample, such as a cell or a tissue sample (such as a tissue section). In some embodiments, the target nucleic acid comprises RNA. In some embodiments, the target nucleic acid is an mRNA. In some embodiments, the circularizable probes are DNA probes.

### II. Probes and Gap Fill

Disclosed herein in some aspects are nucleic acid probes (e.g., full-length and less-than-full-length circularizable probes) that are introduced into a cell or used to otherwise contact a biological sample such as a tissue sample or a sample comprising cells. In some aspects, the nucleic acid probes are circularizable probes each comprising a hybridization region configured to bind to a target sequence in the target nucleic acid. In some aspects, the target nucleic acid comprises RNA and the circularizable probes comprise DNA and/or RNA. In some aspects, the circularizable probes comprise a hybridization region that is a split hybridization region. For example, the hybridization region comprises a first hybridization sequence at the 5′ end of the probe and the second hybridization sequence at the 3′ end of the probe.

In some embodiments, one or more probes in the plurality of circularizable probes comprise a non-phosphorylated 5′ end. In some embodiments, a plurality of full-length circularizable probes in the plurality of circularizable probes comprise a non-phosphorylated 5′ end. In some embodiments, a plurality of less-than-full-length circularizable probes in the plurality of circularizable probes lack phosphorylated 5′ ends. In some cases, one or more probes in the plurality of circularizable probes lacks phosphorylated 5′ ends and the plurality of circularized probes also comprises some probes that comprise a phosphorylated 5′ end.

In some aspects, the methods disclosed herein involve the use of a plurality of circularizable probes that each bind (e.g., hybridize) to different molecules of a target nucleic acid, such as an RNA molecule, wherein after hybridization of the circularizable probe to the target nucleic acid, a gap fill reaction is performed to extend one or more less-than-full-length circularizable probes. In some embodiments, the assays disclosed herein are ligation dependent and only probes that are available for a ligation reaction by having 5′ and 3′ ends that are positioned for ligation are detected. In some embodiments, the ligatable probes are ligated by joining the ends of the probe together. In some embodiments, a circularizable probe ligated to form a circular template (e.g., a DNA probe that directly binds to an RNA target) is amplified through rolling circle amplification.

In some aspects, “binding” as used herein refers to the coupling between two or more nucleic acids, e.g., oligonucleotides and/or polynucleotides. In some embodiments, the binding is indirect binding. In some embodiments, the binding is direct (e.g., binding comprising direct hybridization of nucleic acid sequences). The nature of the binding may vary. In some instances, a first nucleic acid sequence directly binds to a second nucleic acid sequence via hybridization of complementary sequences. In some instances, a first nucleic acid sequence indirectly binds to a second nucleic acid sequence via one or more intermediate nucleic acids. For example, an intermediate nucleic acid comprises a first region that binds to the first nucleic acid sequence and has a second region for binding to the second nucleic acid sequence, thereby forming a complex comprising the first and second nucleic acid sequences.

The probes may comprise any one of a variety of entities that can hybridize to a nucleic acid, typically by Watson-Crick base pairing, such as DNA, RNA, LNA, PNA, etc., depending on the application. In some embodiments, the circularizable probe(s) comprise a hybridization region that is able to bind to at least a portion of a target nucleic acid. The nucleic acid probe may be able to bind to a specific target nucleic acid (e.g., an mRNA, or other nucleic acids as discussed herein). In some embodiments, the nucleic acid probes are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for phosphorylation.

In some embodiments, a circularizable probe comprises one, two, three, four, or more ribonucleotides. In some embodiments, a circularizable probe disclosed herein comprises one, two, three, four, or more ribonucleotides in a DNA backbone. In some embodiments, the one or more ribonucleotides are at and/or near a 3′ end of the circularizable probe. The circularizable probe may comprise an optional 3′ RNA base.

Any suitable circularizable probe may be used to generate the RCA template which is used to generate the RCA product. In some embodiments, a circularizable probe is in the form of a linear molecule which may be circularized by ligating the ends together directly or indirectly, e.g., to each other, or to the respective ends of an intervening (“gap”) oligonucleotide or to an extended 3′ end of the circularizable probe. A circularizable probe may be ligated at the ends (e.g., including an extended gap filled end) to form a circular template. In some instances, a RCA template is circularizable and it is circularized by ligation prior to RCA. Ligation may be templated using a ligation template, and in the case of padlock probes, the target analyte provides the ligation template, or it may be separately provided. The circularizable RCA template comprises at its respective 3′ and 5′ ends regions of complementarity to corresponding cognate complementary regions, which may be adjacent (e.g., directly adjacent) where the ends are directly ligated to each other, or non-adjacent, with an intervening “gap” sequence (e.g., as described in Section II.(ii)) where indirect ligation is to take place.

Provided herein are methods involving the use of plurality of circularizable probes (e.g., padlock probes) for analyzing a target nucleic acid present in a cell or a biological sample, such as a tissue sample. Also provided are probes, sets of probes, compositions, kits, systems and devices for use in accordance with the provided methods. In some aspects, the provided methods and systems can be applied to detect, image, quantitate, or determine the presence or absence of one or more target nucleic acid(s) or portions thereof. In some aspects, the provided methods can be applied to detect, image, quantitate, or determine the sequence of one or more target nucleic acid(s).

In some aspects, a target nucleic acid disclosed herein comprises any polynucleotide nucleic acid molecule (e.g., DNA molecule; RNA molecule, modified nucleic acid, etc.) for assessment in accordance with the provided embodiments, such as a polynucleotide present in a cell. In some embodiments, the target nucleic acid is a coding RNA (e.g., mRNA). In some embodiments, the target nucleic acid is a single RNA molecule. In other embodiments, the target may be at least one RNA molecule, e.g., a group of 2, 3, 4, 5, 6 or more RNA molecules. These RNA molecules may differ in molecule type, and/or may differ in sequence. In some embodiments, the target nucleic acid is, for example, a non-coding RNA (e.g., tRNA, rRNA, microRNA (miRNA), mature miRNA or immature miRNA). In some embodiments, the target nucleic acid is a splice variant of an RNA molecule (e.g., mRNA, pre-mRNA, etc.) in the context of a cell. A suitable target nucleic acid can therefore be an unspliced RNA (e.g., pre-mRNA, mRNA), a partially spliced RNA, or a fully spliced RNA, etc. Target nucleic acids of interest may be variably expressed, e.g., have a differing abundance, within a cell population, wherein the methods of the present disclosure allow profiling and comparison of the expression levels of nucleic acids, comprising but not limited to, RNA transcripts, in individual cells. A target nucleic acid can also be a DNA molecule, e.g., a denatured genomic, viral, plasmid, etc. For example, the methods can be used to detect copy number variants, e.g., in a cancer cell population in which a target nucleic acid is present at different abundance in the genome of cells in the population; a virus-infected cells to determine the virus load and kinetics, and the like.

In some aspects, the methods provided herein are used to analyze a target nucleic acid, e.g., a messenger RNA molecule. In some embodiments, the target nucleic acid is an endogenous nucleic acid present in a biological sample. In some embodiments, molecules of the target nucleic acid are located at a plurality of locations in the biological sample. In some embodiments, the target nucleic acid is present in a cell in a tissue, in a cell pellet, in a cell block, or from a tissue sample (e.g., a tissue section). In some embodiments, the tissue sample is an intact tissue sample or a non-homogenized tissue sample. In some embodiments, the tissue sample is a fresh tissue sample. In some embodiments, the tissue has previously been processed, e.g., fixed, embedded, frozen, or permeabilized (e.g., as described in Section V).

In some aspects, the provided embodiments can be employed for in situ detection and/or sequencing of a target nucleic acid in a cell, e.g., in cells of a biological sample or a sample derived from a biological sample, such as a tissue section on a solid support, such as on a transparent slide.

In some aspects, the methods disclosed herein involve the use of a plurality of circularizable probes that hybridize to a target region of a target nucleic acid, such as an RNA molecule, wherein each circularizable probe is configured to bind to the same target region of a target nucleic acid. For example, as shown in FIG. 2A, a plurality of circularizable probes is synthesized as a plurality of linear nucleic acid strands. In some instances, the synthesis of the oligonucleotide begins at the 3′ end and proceeds to the 5′ end. In some embodiments, a plurality of circularizable probes is synthesized to generate a full-length product (e.g., of n nucleotides in length) that comprises the full first hybridization sequence and full second hybridization sequence for binding to the target region of the target nucleic acid and the full-length circularizable probe is modified with a 5′ phosphate. In some cases, the oligonucleotide synthesis process generates some less-than-full-length circularizable probes, e.g., nucleic acid strands that are short by at least one nucleotide compared to the full-length product. In some instances, the less-than-full-length circularizable probes also are not modified with a 5′ phosphate. As shown in FIG. 2A, the full-length probe (n nucleotides in length) is a single oligonucleotide strand (e.g., a circularizable probe) comprising the first hybridization sequence and the second hybridization sequence at the 5′ and 3′ ends of the circularizable probe. Upon hybridization, the ends of a circularizable probe that is full-length is capable of being ligated together (e.g., are positioned by the hybridization to allow for ligation). A less-than-full-length circularizable probe compared to the full-length circularizable probe has a gap (x nucleotides) between the ends of the less-than-full-length circularizable probe upon hybridization to the target nucleic acid. To fill the gap, the 5′ and/or 3′ end of the less-than-full-length circularizable probe that is short by at least one nucleotide is extended using the target nucleic acid as a template. In some instances, the gap at the 3′ end of the less-than-full-length circularizable probe is filled by extending the 3′ end of the less-than-full-length circularizable probe using a polymerase. After the gap is filled, the ends of the probe are ligated using a ligase (such as a T4 DNA ligase, a PBCV-1 DNA ligase, or a T4 RNA ligase 2 (T4 Rnl2)). In some embodiments, the plurality of circularizable probes comprising fill length and gap filled less-than-full-length circularizable probes are ligated and amplified to generate a rolling circle amplification product (RCA) and then detected.

In some embodiments, a plurality of circularized probes (e.g., a plurality of less-than-full-length circularizable probes) is amplified through rolling circle amplification. In some embodiments, a less-than-full-length circularizable probe is gap filled, circularized, and amplified. In some embodiments, the ligation uses a target nucleic acid as a template. In some embodiments, the target nucleic acid is an RNA. In some embodiments, the plurality of circularizable probes contain one or more barcodes. In some embodiments, the plurality of circularizable probes for targeting the same target nucleic acid comprises the same barcode(s) corresponding to the same target nucleic acid. In some embodiments, one or more barcodes are indicative of a sequence in the target nucleic acid.

In some aspects, the provided methods involve analyzing, e.g., detecting or determining, one or more sequences present in the plurality of circularizable probes and/or in an amplification product, such as in an amplification product of a circularized probe, which may comprise one or more barcode sequences. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a target nucleic acid. In some embodiments, the analysis is used to correlate a sequence detected in an amplification product to a circularizable probe (e.g., via one or more barcodes). In some embodiments, the detection of a sequence in an amplification product provides information regarding the location and/or identity of an associated target nucleic acid (e.g., hybridized by the probe(s)) in a sample. In some embodiments, due to rescue of less-than-full-length circularizable probes that are short by at least one nucleotide compared to a full-length probe, performing a gap fill reaction on the plurality of circularizable probes specific achieves sensitive detection of signals at locations indicative of a target nucleic acid. In particular embodiments, the amplification product is an in situ rolling circle amplification (RCA) product of a circularized probe.

In some embodiments, provided herein are methods for assessing one or more target nucleic acids, such as a plurality of different mRNAs, in a biological sample, such as a cell or a tissue sample (such as a tissue section). In some instances, a first plurality of circularizable probes are for detecting a first target nucleic acid and a second plurality of circularizable probes are for detecting a second target nucleic acid, wherein the first and second target nucleic acids have different identities (e.g., mRNA transcripts associated with different genes). In some embodiments, a first and second target nucleic acid are two different RNA transcripts.

In some embodiments, a target nucleic acid comprises DNA. In some embodiments, the target nucleic acid comprises RNA. In some embodiments, the target nucleic acid comprises mRNA. In some embodiments, the circularizable probe comprises DNA. In some embodiments, the target nucleic acid is RNA and the circularizable probe comprises DNA.

In some aspects, the provided methods involve a step of contacting, or hybridizing, a plurality of circularizable probes, such as any one of the probes described herein, to a cell or a sample containing a target nucleic acid with a target region. In some aspects, the provided methods comprise contacting the plurality of circularizable probes with a kinase to phosphorylate one or more ends of the plurality of circularizable probes. In some aspects, the provided methods comprise contacting the cell or tissue sample with an enzyme to fill the gap of a circularizable probe of the plurality of circularizable probes. In some aspects, the provided methods comprise one or more steps of ligating the ligatable ends of a circularizable probe to form a circularized probe. In some aspects, the one or more steps of ligating the ligatable ends of circularizable probes, comprise ligating the ligatable ends of a circularizable probe that is gap filled after hybridization to the target nucleic acid. In some aspects, the provided methods involve a step of amplifying a sequence of the circularizable probes (e.g., a padlock probe), to generate amplification products. In some aspects, the provided methods comprise detecting and/or determining the sequence of all or a portion of the amplification product (for example, of one or more barcodes contained in the amplification product). In some aspects, the provided methods involve performing one or more of the steps described herein, simultaneously and/or sequentially.

In some embodiments, a circularizable probe herein comprises from the 3′ to 5′ direction: a first hybridization region, a barcode, and a second hybridization region. In some embodiments, a circularizable probe herein comprises from the 5′ to 3′ direction: a first hybridization region, a barcode, and a second hybridization region. The first and second hybridization regions can form a split hybridization region, and upon probe hybridization to a target nucleic acid, the first and second hybridization regions each comprises an end that is juxtaposed for ligation to each other (with or without gap filling prior to the ligation) to circularize the circularizable probe. In some embodiments, a less-than-full-length circularizable probe is short by at least one nucleotide at the first hybridization sequence at the 5′ end of the circularized probe compared to a corresponding hybridization sequence in the full-length probe. In some embodiments, a less-than-full-length circularizable probe is short by at least one nucleotide at the first hybridization sequence at the 3′ end of the circularized probe compared to a corresponding hybridization sequence in the full-length probe.

### (i) Kinase Treatment

In some embodiments, at least one probe of the plurality of circularizable probes comprises a non-phosphorylated 5′ end. In some embodiments, a plurality of less-than-full-length circularizable probes comprises a non-phosphorylated 5′ end. In some embodiments, at least 5%, 10%, 15%, or 20% of probes in the plurality of circularizable probes are lacking a 5′ phosphate. In some embodiments, between 5% to 50%, between 10% to 50%, between 15% to 50 percent, between 20% to 50%, between 30% to 50%, between 40% to 50%, between 10% to 40%, between 10% to 30%, between 10% to 20%, between 15% to 40%, between 20% to 40%, between 15% to 30%, between 20% to 30%, or between 30% to 50% of probes in the plurality of circularizable probes are lacking a 5′ phosphate. In some embodiments, the less-than-full-length circularizable probes are missing at least one nucleotide at the 5′ terminus of the first hybridization sequence compared to the 5′ first hybridization sequence of the full-length probe. In some cases, the at least one nucleotide missing from a less-than-full-length circularizable probe prevents the 5′ phosphate from being added at the end of oligonucleotide synthesis.

In some instances, the method comprises using a kinase to phosphorylate the non-phosphorylated 5′ end of the at least one probe of the plurality of circularizable probes prior to contacting the biological sample with the plurality of circularizable probes. In some embodiments, a plurality of circularizable probes is contacted with a kinase to add a 5′ phosphate. In some aspects, the kinase catalyzes the transfer of the gamma-phosphate from ATP to the 5′—OH group of the nucleic acid circularizable probe. As shown in FIG. 2B, a plurality of circularizable probes is treated with a kinase to add a phosphate to the end of the less-than-full-length circularizable probes lacking a 5′ phosphate. In some embodiments, in vitro phosphorylation of the plurality of circularizable probes is performed. In some embodiments, phosphorylation of the plurality of circularizable probes is performed after the circularizable probes are contacted with the biological sample. In some embodiments, phosphorylation of the plurality of circularizable probes is performed after the circularizable probes are bound to target nucleic acids. In some embodiments, a plurality of circularizable probes is contacted with a kinase to add a 5′ phosphate after being added to the biological sample. In some embodiments, a plurality of circularizable probes is contacted with a kinase to add a 5′ phosphate after gap filling at least some portion of the plurality of circularizable probes. After contacting with the kinase, the plurality of circularizable probes comprising a phosphate at the 5′ end of the probe are available for ligation (e.g., circularization).

In some embodiments, the plurality of circularizable probes is contacted with a kinase to phosphorylate the 5′ ends. In some embodiments, the method comprises contacting the biological sample with a polynucleotide kinase (PNK). In some embodiments, the method comprises contacting the plurality of circularizable probes with a polynucleotide kinase (PNK). In some embodiments, the method comprises contacting the plurality of circularizable probes with a polynucleotide kinase (PNK) in vitro (e.g., prior to contacting the plurality of circularizable probes with the biological sample). In some embodiments, the plurality of circularizable probes is in the biological sample when the PNK is provided. In some embodiments, the plurality of circularizable probes is contacted with at least any of 0.05 U, 0.1 U, 0.5 U, 1 U, 5 U, 10 U, 50 U, 100 U, 200 U, 300 U, 400 U, 500 U, 600 U, 700 U, 800 U, 900 U, or 1000 U of a kinase. In some embodiments, the plurality of circularizable probes is contacted with about 0.1 U/μL to about 5 U/μL, about 0.1 U/μL to about 2 U/μL, about 0.1 U/μL to about 1 U/μL, about 0.2 U/μL to about 5 U/μL, about 0.2 U/μL to about 2 U/μL, about 0.2 U/μL to about 1 U/μL, about 0.5 U/μL to about 5 U/μL, 0.5 U/μL to about 2 U/μL, about 0.5 U/μL to about 1 U/μL of a kinase. In some embodiments, the plurality of circularizable probes is contacted with about 0.5 U/μL of a kinase. In some embodiments, the plurality of circularizable probes is contacted with about 0.5 U/μL of a kinase in vitro prior to contacting the plurality of circularizable probes with the biological sample. In some embodiments, at least any of 0.05 U/μL, 0.1 U/μL, 0.5 U/μL, 1 U/μL, 2 U/μL, 3 U/μL, 4 U/μL, 5 U/μL, 6 U/μL, 7 U/μL, 8 U/UL, 9 U/μL, 10 U/μL, 20 U/μL, 30 U/μL, 40 U/μL, or 50 U/μL of a polynucleotide kinase as the final concentration is used to treat a plurality of circularizable probes. One unit of PNK activity is defined as the amount of enzyme (measured in units, U) that will catalyze the transfer of 1 nmol of phosphate from ATP to the 5′—OH end of a polynucleotide in 30 minutes at an optimum temperature for the enzyme, usually 37° C. In some embodiments, the PNK transfers the gamma phosphate group from adenosine triphosphate (ATP) to the 5′ hydroxyl termini of DNA or RNA (e.g., probes described herein). In some embodiments, the kinase is an enzyme from the family of transferases that transfer phosphorus-containing groups to alcohol groups (phosphotransferases). Systematically, the kinase class may be from the enzyme class known as ATP: 5′-diphospho polynucleotide 5′-phosphotransferase. In some embodiments, the kinase is a T4 PNK or a variant thereof. In some embodiments, the kinase is a T7 PNK or a variant thereof.

In some embodiments, phosphorylation comprises incubating the plurality of circularizable probes with the kinase (e.g., polynucleotide kinase (PNK)). In some embodiments, the method comprises incubating the plurality of circularizable probes with the kinase for at least 20 minutes, at least 30 minutes, at least 35 minutes, at least 40 minutes, at least 45 minutes, at least 50 minutes, at least 60 minutes, at least 80 minutes, at least 100 minutes, or at least 120 minutes. In some embodiments, the incubation with the kinase is performed for 10-60 minutes, 10-45 minutes, 10-30 minutes, 15-60 minutes, 15-45 minutes, 15-30 minutes, 20-45 minutes, 20 -120 minutes, 30-120 minutes, 30-60 minutes, or 30-90 minutes. In some embodiments, the incubation with kinase is performed at 30° C. to 40° C., e.g., at 37° C. In some embodiments, the plurality of circularizable probes is incubated with the kinase for 30 minutes.

In some embodiments, at least one probe of the plurality of circularizable probes comprises a non-ligatable end. In some embodiments, less-than-full-length circularizable probes of the plurality of circularizable probes comprise a non-ligatable end. In some instances, the kinase treatment converts one or more non-ligatable ends to an end with a 5′ phosphate. In some embodiments, a kinase treatment step is before and/or after one or more wash steps for phosphorylation performed in the biological sample. In some embodiments, a kinase treatment step is after one or more wash steps of the biological sample. In some embodiments, the wash step comprises removing unbound probes not hybridized to the target nucleic acid from the sample. In some embodiments, the method comprises performing a wash to remove unbound circularizable probes prior to contacting the cell or tissue sample with the kinase. In some instances, the wash is performed prior to kinase treatment with a buffer that does not contain phosphate. In some instances, the wash is performed with a saline-sodium citrate (SSC) buffer. For example, the wash may be performed using a ligation buffer comprising a salt and ATP. In some cases, the wash is performed with a buffer that is between about pH 7.5 to about pH 8.5. In some cases, the wash is performed with a buffer that is at about pH 8.0. In some embodiments, the sample is washed prior to kinase treatment with a ligation buffer. In some embodiments, the wash buffer comprises MgCl₂, KCI, ATP, DTT, glycerol and BSA.

In some embodiments, the kinase is provided in excess to phosphorylate the one or more ends of the circularizable probes hybridized in the sample. In some embodiments, the kinase is not endogenous to the sample, e.g., the kinase is exogenously provided to the sample where the circularizable probes are hybridized. In some embodiments, the kinase is not tethered to any molecules of the circularizable probes.

### (ii) Gap Filling

In some embodiments, provided herein is a method for analyzing a biological sample, comprising contacting the biological sample with a plurality of circularizable probes. In some embodiments, a circularizable probe of the plurality of circularizable probes comprises a linear oligonucleotide sequence that, upon hybridization to a target nucleic acid, such as an RNA molecule, forms a probe that can be circularized. In some instances, a probe is circularized via ligation (or primer extension followed by ligation) using the target nucleic acid (e.g., target RNA) as a template. In some embodiments, the plurality of circularizable probes comprises a full-length circularizable probe and a less-than-full-length circularizable probe. For example, the plurality of circularizable probes comprises at least one probe of the plurality of circularizable probes that is different in length than other probes in the plurality. In some instances, upon hybridization to the target nucleic acid, a gap is formed between a first hybridization sequence at the 5′ end and a second hybridization sequence at the 3′ end of the less-than-full-length circularizable probe. In some instances, this gap between the ends of the less-than-full-length circularizable probe that is formed upon hybridization is filled.

In some aspects, a circularizable probe of the plurality of circularizable probes comprises a hybridization region that hybridizes to the target region in the target nucleic acid. In some aspects, the hybridization region comprises a first hybridization sequence at the 5′ end of the probe molecule and a second hybridization sequence at the 3′ end of the probe molecule. Once hybridized to the complementary sequences in the target nucleic acid, the first and second hybridization sequences are positioned with a gap of about 5, 4, 3, 2, or 1 nucleotides away. In some embodiments, the 5′ end of the circularizable probe is phosphorylated by a kinase (e.g., as described in Section II (i)).

In some embodiments, a gap of a less-than-full-length circularizable probe hybridized to the target nucleic acid is filled by extending a 3′ end of the less-than-full-length circularizable probe. In some embodiments, a polymerase is used to extend the 3′ end using the target nucleic acid as a template, thereby filling the gap using nucleotide sequence in the target nucleic acid. In some embodiments, gap filling by the polymerase incorporates nucleotides residues into the circularizable probe, and the incorporated nucleotide sequence is complementary to the gap sequence or a portion thereof in the target nucleic acid. In some embodiments, a 3′ end and a 5′ end of the extended less-than-full-length circularizable probe is ligated using the target nucleic acid (e.g., RNA) as a template after gap filling. In some embodiments, the ligation of the 3′ end and the 5′ end is preceded by gap filling. In some instances, filling the gap of a less-than-full-length circularizable probe generates an extended 5′ end of the first hybridization region of the less-than-full-length circularizable probe and a ligase ligates the extended 5′ end of the first hybridization region to the 3′ end of the second hybridization region using the target nucleic acid as a template.

In some embodiments, a gap of at least 1, 2, 3, 4, 5, or more nucleotides between the first hybridization sequence and the second hybridization sequence hybridized to a target nucleic acid is filled prior to ligation. In some embodiments, a gap of 1-50 nucleotides, 1-40 nucleotides, 1-30 nucleotides, 1-20 nucleotides, 1-10 nucleotides, 1-5 nucleotides, 2-50 nucleotides, 2-40 nucleotides, 2-30 nucleotides, 2-20 nucleotides, 2-10 nucleotides, 2-5 nucleotides, 5-50 nucleotides, 5-40 nucleotides, 5-30 nucleotides, 5-20 nucleotides, 5-10 nucleotides, 10-50 nucleotides, 10-40 nucleotides, 10-30 nucleotides, or 10-20 nucleotides between the first hybridization sequence and the second hybridization sequence hybridized to a target nucleic acid is filled prior to ligation. In some embodiments, a gap of 10-50 nucleotides between the first hybridization sequence and the second hybridization sequence hybridized to a target nucleic acid is filled prior to ligation. In specific embodiments, the gap is a gap of about 1, 2, 3, 4, 5, 6, 7, 8, 9 or 10 or more nucleotides, of any integer (or range of integers) of nucleotides in between the indicated values herein. In some embodiments, a gap of no more than 20, no more than 30, no more than 40, or no more than 50 nucleotides between the first hybridization sequence and the second hybridization sequence hybridized to a target nucleic acid is filled. In some cases, the gap is a gap of 1 to 60 nucleotides or a gap of 1 to 40 nucleotides or a gap of 3 to 40 nucleotides. In some embodiments, the ligation herein is preceded by gap filling of at least some probes of plurality of circularizable probes contacted with a biological sample, wherein the plurality of circularizable probes target the same sequence of a target nucleic acid.

In some embodiments, performing the gap-fill reaction comprises using a gap-fill polymerase (e.g., DNA polymerase) to extend an end of the less-than-full-length circularizable probe using the target RNA as a template to generate an extended probe. FIG. 2B shows a workflow for generating a plurality of circularized probes from circularizable probes comprising a full-length oligonucleotide (Probe 2) and a less-than-full-length circularizable probe (Probe 1) compared to the full-length oligonucleotide. The synthesized probe oligonucleotides are treated with a phosphate modification and contacted with the target nucleic acid. Unlike Probe 2, which is a full-length circularizable probe that is positioned for ligation of the adjacent ends upon hybridization to the target nucleic acid, Probe 1 as shown hybridizes to the complementary sequences in the target nucleic acid, and the first and second hybridization sequences are positioned with a gap of at least 1 nucleotides and is not ligatable upon hybridization with this gap. In some embodiments, the gap of Probe 1 is filled and an extended end of Probe 1 is ligated to another end of the probe after the gap is filled.

In some embodiments, the gap is filled using a polymerase that has no or little strand displacement activity. In some embodiments, the gap is filled using a gap-fill polymerase that incorporates one or more deoxyribonucleotide residues and/or one or more ribonucleotide residues into a 3′ end of the circularizable probe. In some instances, a gap filled circularizable probe comprises one or more ribonucleotide residues at and/or near its 3′ end.

In some instances, the gap filling is performed using a polymerase (e.g., DNA polymerase) in the presence of appropriate dNTPs and other cofactors, under isothermal conditions or non-isothermal conditions. Suitable DNA polymerases include but are not limited to: E. coli DNA polymerase I, Bsu DNA polymerase, Bst DNA polymerase, Taq DNA polymerase, Thermococcus gorgonarius polymerase (Tgo DNA polymerase), VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes. In some instances, the gap filling is performed using a Sulfolobus DNA Polymerase. In some instances, the gap filling is performed using a Sulfolobus DNA Polymerase IV. In some instances, the gap filling is performed using a Klenow fragment. In some instances, the gap filling is performed using a Taq DNA polymerase or a modification or fragment thereof. In some instances, the gap filling is performed using a Hemo KlenTaq® DNA polymerase.

In some instances, the gap filling is performed using a DNA polymerase capable of incorporating at least about 1, at least about 2, at least about 3, at least about 4, at least about 5, at least about 6, at least about 7, at least about 8, at least about 9, at least about 10, at least about 15, at least about 20, at least about 25, at least about 30, at least about 40, or at least about 50 nucleotides in a single binding event before dissociating from the target nucleic acid molecule.

Incorporation of the correct nucleotides to a growing strand of DNA, as determined by the template, is known as sequence fidelity. In some embodiments, a high fidelity DNA polymerase is used for gap filling. In some instances, the gap filling is performed using a polymerase having no or limited strand displacement activity, such that an extended 3′ region of the circularizable probe does not displace the 5′ region hybridized to the nucleic acid molecule. For example, T4 and T7 DNA Polymerases lack strand displacement activity and can be used for this purpose. In some embodiments, especially where the target nucleic acid is RNA, the polymerase can be a reverse transcriptase. Reverse transcriptases having reduced strand displacement activity can be used, see, e.g., Martín-Alonso et al., ACS Infect. Dis. 2020, 6, 5, 1140-1153, which is incorporated herein by reference in its entirety.

In some embodiments, the method does not comprise filling a gap between the first and second hybridization sequences of a full-length circularizable probe. In some embodiments, a full-length probe with the 5′ terminal nucleotide and the 3′ terminal nucleotide of the full-length circularizable probe hybridized to adjacent nucleotides in the target region is not gap filled. In some instances, the biological sample is contacted with a plurality of circularizable probes comprising a full-length circularizable probe and a less-than-full-length circularizable probe, wherein the plurality of circularizable probes target the same target region of a target nucleic acid, and the full-length probe is not configured to be gap filled.

### (iii) Ligation and Amplification

In some aspects, after gap filling any probes in the plurality of circularizable probes that require extension to generate ligatable ends positioned for ligation, the method further comprises one or more steps such as ligation, extension and/or amplification of the plurality of circularizable probes hybridized to the target nucleic acids. In some instances, the ligation to form a plurality of circularized probes is performed in the same reaction as the gap filling as described in Section II. (ii). In some embodiments, the methods of the present disclosure comprise performing rolling circle amplification in the presence of a target nucleic acid of interest.

In some embodiments, some probes of the plurality of circularizable probes (e.g., less-than-full-length circularizable probes) are gap filled before the 3′ end and the 5′ end are ligated and some (e.g., full-length probes) are ligated without gap filling. In some instances, two separate ligations are performed. In some instances, forming circularized probes from the full-length circularizable probe and forming circularized probes from the less-than-full-length circularizable probes are performed separately. In some instances, forming circularized probes from the full-length circularizable probe and forming circularized probes from the less-than-full-length circularizable probes are performed at different times. In some instances, forming circularized probes from the full-length circularizable probe and forming circularized probes from the less-than-full-length circularizable probes are performed in the same step. In some embodiments, the ligation to generate circularized probes for probes that are gap filled and probes that are not gap filled are distinct. In some embodiments, the ligation to generate circularized probes from gap filled less-than-full-length circularizable probes is performed after ligation of probes (e.g., full-length circularizable probes) that are not gap filled. In some embodiments, for a plurality of circularizable probes in a mixture comprising full-length circularizable probes and less-than-full-length circularizable probes, a first ligation is performed (e.g., to circularize full-length circularizable probes), followed by gap fill of any less-than-full-length circularizable probes, and then a second ligation is performed after the gap fill (e.g., to circularize extended less-than-full-length circularizable probes).

In some embodiments, provided herein is a method comprising contacting a biological sample with a plurality of circularizable probes comprising a full-length circularizable probe and a less-than-full-length circularizable probe, wherein the plurality of circularizable probes target the same target region of a target nucleic acid, and the circularizable probes of the plurality of circularizable probes comprise a first hybridization sequence at the 5′ end and a second hybridization sequence at the 3′ end, wherein the first hybridization sequence and the second hybridization sequence hybridize to a sequence of at least a portion of the target region, wherein the 5′ terminal nucleotide and the 3′ terminal nucleotide of the full-length circularizable probe hybridize to adjacent nucleotides in the target region, and wherein the 5′ terminal nucleotide and the 3′ terminal nucleotide of the less-than-full-length circularizable probe hybridize to non-adjacent nucleotides in the target region such that there is a gap (e.g., of one, two, three, four, or more nucleotides) between the first hybridization sequence and the second hybridization sequence of the less-than-full-length circularizable probe; contacting the biological sample with a first ligase; filling a gap between the first hybridization sequence and the second hybridization sequence of the less-than-full-length circularizable probe to form a gap-filled circularizable probe; and contacting the biological sample with an additional ligase, thereby forming a plurality of circularized probes from the full-length circularizable probe and the gap-filled circularizable probe in the biological sample. In some embodiments, the first ligase is provided during or prior to filling the gap between the first hybridization sequence and the second hybridization sequence. In some instances, the first ligase and the additional ligase are the same type of ligase.

In some embodiments, the biological sample is contacted with a ligase during or prior to filling the gap between the first hybridization sequence and the second hybridization sequence. In some embodiments, the full-length circularizable probes are ligated prior to filling the gap between the first hybridization sequence and the second hybridization sequence of the less-than-full-length circularizable probe. In some embodiments, the biological sample is contacted with an additional ligase after filling the gap between the first hybridization sequence and the second hybridization sequence to form the plurality of circularized probes. In some embodiments, the additional ligase is the same ligase (e.g., additional molecules of the same ligase) as the ligase used to form circularized probes from the full-length circularizable probes. In some embodiments, the biological sample is contacted with a ligase after filling the gap between the first hybridization sequence and the second hybridization sequence.

In some embodiments, the plurality of circularizable probes are incubated with the ligase. In some embodiment, two separate ligation reactions are performed. In some instances, each ligation incubation (e.g., a first ligation incubation and a second ligation incubation) is performed for at least 20 minutes. In some instances, each ligation incubation is performed for no more than 45 minutes. In some instances, each ligation incubation is performed for no more than 60 minutes. In some instances, each ligation incubation is performed for no more than 120 minutes. In some embodiments, the method comprises incubating the plurality of circularizable probes with the ligase for at least 20 minutes, at least 30 minutes, at least 35 minutes, at least 40 minutes, at least 45 minutes, at least 50 minutes, at least 60 minutes, at least 80 minutes, at least 100 minutes, or at least 120 minutes. In some embodiments, the incubation with the ligase is performed for 10-20 minutes, 10-30 minutes, 10-40 minutes, 15-60 minutes, 15-45 minutes, 15-30 minutes, 20-45 minutes, 20 -120 minutes, 30-120 minutes, 30-60 minutes, or 30-90 minutes. In some embodiments, the incubation with ligase is performed at 30° C. to 40° C., e.g., at 37° C. In some embodiments, the plurality of circularizable probes is incubated with the ligase for two separate 30 minute incubations.

In some embodiments, the method comprises using a circularizable construct hybridized to the target nucleic acid to generate a product (e.g., comprising a sequence associated with the target nucleic acid). In some aspects, the product is generated using rolling circle amplification (RCA). In some embodiments, the method comprises ligating the ends of a circularizable probe hybridized to the target RNA to form a circularized probe. In some embodiments, the method comprises ligating the ends of a plurality of circularizable probes hybridized to the target RNA to form a plurality of circularized probes. In some instances, the method further comprises generating a plurality of RCA products using the plurality of circularizable probes.

In some embodiments, the RCA comprises a linear RCA. In some embodiments, the RCA comprises a branched RCA. In some embodiments, the RCA comprises a dendritic RCA. In some embodiments, the RCA comprises any combination of the foregoing. In any one of the embodiments herein, the method can further comprise detecting a signal associated with the rolling circle amplification product in the biological sample. In some embodiments, a plurality of ligated circularizable probes (e.g., circularized probes) are detected. In some cases, RCA is not performed.

In some embodiments, the circular construct (e.g., circularized probes) is formed using ligation. In some embodiments, the ligated probe is generated using the target nucleic acid as template. In some embodiments, the ligation is a DNA-DNA templated ligation. In some embodiments, the ligation is an RNA-RNA templated ligation. In some embodiments, the ligation is a RNA-DNA templated ligation, for instance ligation of DNA probes is dependent on the RNA template.

In some embodiments, ligating the first ligatable end to a second ligatable end in the circularizable probe may comprise enzymatic ligation, chemical ligation, template dependent ligation, and/or template independent ligation. For example, a first hybridization sequence of the circularizable probe comprises a first ligatable end and a second hybridization sequence of the circularizable probe comprises a second ligatable end. In some instances, a first hybridization sequence of the circularizable probe extended by gap fill comprises a first ligatable end and a second hybridization sequence of the circularizable probe comprises a second ligatable end. In some embodiments, ligating the first ligatable end to a second ligatable end in the circularizable probe is a template dependent ligation, for example, wherein the ligation depends on hybridization to the target nucleic acid. In some instances, the ligation comprises using a ligase having an RNA-templated DNA ligase activity and/or an RNA-templated RNA ligase activity. In some embodiments, the enzymatic ligation involves use of a ligase (e.g., an RNA ligase, a DNA ligase). Ligases comprise ATP-dependent double-strand polynucleotide ligases, NAD-i-dependent double-strand DNA or RNA ligases and single-strand polynucleotide ligases, for example any one of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In any one of the embodiments herein, the ligation comprises using a ligase selected from the group consisting of a Chlorella virus DNA ligase (PBCV DNA ligase), a T4 RNA ligase, a T4 DNA ligase, and a single-stranded DNA (ssDNA) ligase. In any one of the embodiments herein, the ligation can comprise using a PBCV-1 DNA ligase or variant or derivative thereof and/or a T4 RNA ligase 2 (T4 Rnl2) or variant or derivative thereof. In some embodiments, the ligase is a T4 RNA ligase. In some embodiments, the ligase comprises a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, a ligation buffer disclosed herein comprises ATP and/or NAD+ as an alternative adenylation donor for nucleic acid ends ligation catalyzed by a ligase.

In some embodiments, the ligation herein is a direct ligation. In some embodiments, full-length circularizable probes are ligated with a direct ligation. In some embodiments, the ligation herein is an indirect ligation. In some embodiments, a direct ligation occurs between ends of the polynucleotides hybridized immediately adjacently to one another to form a substrate for a ligase enzyme. In some embodiments, the direct ligation is an intramolecular ligation. In some embodiments, an indirect ligation occurs when the ends of the polynucleotides hybridize non-adjacently to one another, (for instance, separated by one or more intervening nucleotides or gaps). In some embodiments, the ends of the polynucleotides are ligated following extension of the 3′ end of a circularizable probe (e.g., by a polymerase) to fill the “gap” corresponding to said intervening nucleotides. In some embodiments, the indirect ligation is an intermolecular ligation.

In some aspects, a high-fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tm) of the DNA strands. This selectively reduces the concentration of hybridized mismatched ligation substrates (e.g., wherein the interrogatory region is not complementary to the region of interest, such that the probe or probes are expected to have a slightly lower Tm around the mismatch) over hybridized fully base-paired ligation substrates (e.g., wherein the interrogatory region is complementary to the region of interest). Thus, high-fidelity ligation can be achieved through a combination of the intrinsic selectivity of the ligase active site and balanced conditions to reduce the incidence of annealed mismatched dsDNA.

In some embodiments, the method comprises prior to ligating the first ligatable end to a second ligatable end in the circularizable probes, removing molecules of the circularizable probes that are not bound to the target nucleic acid from the biological sample.

Following formation of the plurality of circularized probes, in some instances, an amplification primer is added. In other instances, the amplification primer is added with the circularizable probes. In some embodiments, the amplification primer is added after contacting the sample with the ligase. In some embodiments, the amplification primer is added after filling the gaps. In some instances, the amplification primer is complementary to the target nucleic acid and the circularizable probe. In some embodiments, an amplification primer is not needed and an extension reaction uses the target nucleic acid as a primer. In some embodiments, a wash is performed to remove any unbound probes, primers, etc. In some embodiments, the wash is a stringency wash. Washes can be performed at any point during the process to remove non-specifically bound probes, probes that have ligated, etc. In some embodiments, the stringency is increased in the hybridization of the circularizable probe to the target nucleic acid, reducing or negating the need of performing a stringency wash.

Upon addition of a DNA polymerase in the presence of appropriate dNTP precursors and other cofactors, the amplification primer or target nucleic acid is elongated by replication of multiple copies of the template (e.g., a concatemer of the template is generated). In some instances, the amplification product is detected using, e.g., the secondary and higher order probes and detection oligonucleotides described herein. In some embodiments, the sequence of the amplicon (e.g., RCA product) or a portion thereof, is determined or otherwise analyzed, for example by using detectably labeled probes and imaging. The sequencing or analysis of the amplification products can comprise sequencing by hybridization, sequencing by ligation, and/or fluorescent in situ sequencing, and/or wherein the in situ hybridization comprises sequential fluorescent in situ hybridization. In some instances, sequencing using, e.g., the secondary and higher order probes and detection oligonucleotides described herein.

In some embodiments, the method comprises generating the plurality of amplification products of the plurality of circularized probes in situ in the biological sample. In some embodiments, the products are generated using rolling circle amplification (RCA). In some embodiments, the RCA comprises a linear RCA, a branched RCA, a dendritic RCA, or any combination thereof. In some embodiments, the product is generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, RCA is performed using a Phi29 DNA polymerase.

In some embodiments, upon addition of a DNA polymerase in the presence of appropriate dNTP precursors and other cofactors, primers are elongated to produce multiple copies of the circular template. This amplification step can utilize isothermal amplification or non-isothermal amplification. Techniques for rolling circle amplification (RCA) include but are not limited to linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29: el 18, 2001; Dean et al. Genome Res. 1 1: 1095-1099, 2001; Schweitzer et al, Nature Biotech. 20:359-365, 2002; U.S. Patent Nos. 6,054,274, 6,291,187, 6,323,009, 6,344,329 and 6,368,801). Exemplary polymerases for use in RCA comprise DNA polymerase such phi 29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) are anchored to a polymer matrix. For example, the polymer matrix comprises a hydrogel. In some embodiments, one or more of the polynucleotide probe(s) are modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465. In some examples, the scaffold also contains modifications or functional groups that can react with or incorporate the modifications or functional groups of the circularizable probe or amplification product. In some examples, the scaffold can comprise oligonucleotides, polymers or chemical groups, to provide a matrix and/or support structures.

In some embodiments, a plurality of amplification products are immobilized within the matrix generally at the location of the nucleic acid being amplified, thereby creating a localized colony of amplicons. In some embodiments, the amplification products are immobilized within the matrix by steric factors. In some embodiments, the amplification products are immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probes and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, an example workflow (as shown in FIG. 2B) comprises contacting a biological sample with a plurality of circularizable probes, wherein the plurality of circularizable probes target the same sequence of a target region of a target nucleic acid, and each circularizable probe of the plurality of circularizable probes comprises a hybridization region comprising a first hybridization sequence at the 5′ end and a second hybridization sequence at the 3′ end. In some embodiments, the plurality of circularizable probes comprise full-length and less-than-full-length circularizable probes. In some embodiments, the first hybridization sequence and the second hybridization sequence are complementary to at least a portion of the target region. As shown in FIGS. 2A and 2B, at least one probe of the plurality of circularizable probes is different in length than other probes in the plurality. In some aspects, the method comprises filling a gap between the first hybridization sequence and the second hybridization sequence of the less-than-full-length circularizable probes and forming a plurality of circularized probes (from the full-length and less-than-full-length circularizable probes) in the biological sample, and amplifying the plurality of circularized probes using rolling circle amplification (RCA) to generate a plurality of RCA products. In some embodiments, the plurality of RCA products is detected in the biological sample. In some embodiments, the plurality of RCA products is detected at a plurality of locations in the biological sample. In some aspects, the plurality of generated RCA products is associated with the same target nucleic acid (e.g., same target mRNA). In some embodiments, one or more signals associated with the plurality of RCA products are detected in the biological sample.

### III. Analytes

The methods and compositions disclosed herein can be used to detect and/or analyze a wide variety of different analytes. In some embodiments, the methods and compositions are for analysis of a plurality of endogenous analytes (and/or products thereof generated in situ) present in a cell or a biological sample, such as a tissue sample. In some aspects, an analyte can include any biological substance, structure, moiety, or component to be analyzed. In some aspects, a target analyte (e.g., a target nucleic acid) disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo-or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

In some embodiments, an analyte herein is endogenous to a biological sample and can include nucleic acid analytes and non-nucleic acid analytes. In some instances, the target analyte is a target nucleic acid. Methods and compositions disclosed herein can be used to analyze nucleic acid analytes (e.g., using a plurality of circularizable probes described in Section II) that directly or indirectly hybridizes to a target nucleic acid).

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes. In some embodiments, any number of molecules of a particular analyte is detected. For example, at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more molecules of a particular analyte are detected.

In any embodiment described herein, the analyte comprises a target sequence. In some embodiments, the target sequence is endogenous to the sample, generated in the sample, added to the sample, or associated with an analyte in the sample. In some embodiments, the target sequence is a single-stranded target sequence (e.g., a mRNA sequence). In some embodiments, the analytes comprise one or more single-stranded target sequences. In some embodiments, provided herein are methods and compositions for analyzing endogenous analytes (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product) thereof. A target sequence for a plurality of circularizable probes disclosed herein may be comprised in any analyte disclose herein, including a plurality of endogenous analytes (e.g., a viral or cellular nucleic acid) or products thereof.

### IV. Detection and Analysis

In some aspects, the provided methods are employed for in situ analysis of target nucleic acids, for example for in situ sequencing or multiplexed analysis in intact tissues or a sample with preserved cellular or tissue structure. In some aspects, the provided methods are used to detect or determine the identity or amount in situ of a target nucleic acid.

In some aspects, after hybridizing the plurality of circularizable probes described in Section II and any one or more further processing steps (e.g., ligation, gap fill, amplification, or any combination thereof), the method further comprises detection a sequence associated with the plurality of circularizable probes hybridized to the target nucleic acids or any products generated therefrom or a derivative thereof. In some embodiments, the method comprises imaging the biological sample to detect the plurality of circularized probes or products thereof. In some embodiments, a sequence of the rolling circle amplification products is detected in situ in the biological sample. In some embodiments, a signal associated with the rolling circle amplification products is detected in situ in the biological sample. In some embodiments, a plurality of signals associated with the plurality of rolling circle amplification products is detected in situ in the biological sample. In some embodiments, the imaging comprises detecting a signal associated with a fluorescently labeled probe that directly or indirectly binds to a rolling circle amplification product of a circularized probe of the plurality of circularizable probes contacted with the biological sample. In some embodiments, the plurality of circularized probes or the plurality of rolling circle amplification products are detected by sequential hybridization, sequencing by hybridization, sequencing by ligation, sequencing by synthesis, sequencing by binding, or a combination thereof.

In some embodiments, a sequence associated with the target nucleic acid or the plurality of circularizable probes comprise one or more barcode sequences or complements thereof. In some embodiments, the sequence of a plurality of rolling circle amplification products comprise one or more barcode sequences or complements thereof. In some embodiments, the one or more barcode sequences comprise a barcode sequence corresponding to the target nucleic acid.

In some aspects, a probe of the plurality of circularizable probes comprise one or more barcode(s), e.g., at least two, three, four, five, six, seven, eight, nine, ten, or more barcodes.

Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or UMI). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, barcodes or complements thereof (e.g., barcode sequences or complements thereof comprised by the probes disclosed herein or products thereof) are analyzed (e.g., detected or sequenced) using any suitable method or technique, including those described herein, such as sequencing by synthesis (SBS), sequencing by ligation (SBL), or sequencing by hybridization (SBH).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4N complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and 20210164039, which are hereby incorporated by reference in their entirety.

In some embodiments, the target nucleic acid is at a plurality of locations in a biological sample and the plurality of circularized probes is generated at the locations in the biological sample, and the plurality of circularized probes and/or the product thereof is detected at the locations in the biological sample.

In some embodiments, the detecting comprises contacting the biological sample with one or more detectably-labeled probes that directly or indirectly hybridize to the rolling circle amplification product, and dehybridizing the one or more detectably-labeled probes from the rolling circle amplification product. In some embodiments, the contacting and dehybridizing are repeated with the one or more detectably-labeled probes and/or one or more other detectably-labeled probes that directly or indirectly hybridize to the rolling circle amplification product. In some embodiments, the detectably-labeled probes directly hybridize to the rolling circle amplification product (e.g., generated as described in Section II).

In some embodiments, the detecting comprises contacting the biological sample with a detectably labeled probe, wherein the detectably labeled probe comprises a hybridization region complementary to one barcode sequence or complement thereof of the one or more barcode sequences or complements thereof, and detecting complexes formed between the one or more barcode sequences or complements thereof and the detectably labeled probe.

In some embodiments, the detecting step comprises contacting the biological sample with one or more intermediate probes that directly or indirectly hybridize to the rolling circle amplification product, wherein the one or more intermediate probes are detectable using one or more detectably-labeled probes. In some embodiments, the detecting step comprises dehybridizing the one or more intermediate probes and/or the one or more detectably-labeled probes from the rolling circle amplification product. In some embodiments, the contacting and dehybridizing are repeated with the one or more intermediate probes, the one or more detectably-labeled probes, one or more other intermediate probes, and/or one or more other detectably-labeled probes.

In some cases, the repeated contacting, detection and dehybridizing steps allows detection of barcode sequences or complements thereof and identification of the corresponding sequences of signal codes (e.g., fluorophore sequences assigned to the corresponding barcode sequences or complements thereof).

In some embodiments, the method comprises detecting the RCA product by hybridizing one or more linear probes to the RCA product. In some embodiments, a linear probe is one that comprises a target recognition sequence (e.g., a sequence complementary to a barcode sequence or subunit thereof in the RCA product) and a sequence that does not hybridize to a target nucleic acid, such as a 5′ overhang, a 3′ overhang, and/or a linker or spacer (which may comprise a nucleic acid sequence or a non-nucleic acid moiety). In some embodiments, the sequence (e.g., the 5′ overhang, 3′ overhang, and/or linker or spacer) is non-hybridizing to the target nucleic acid but may hybridize to one another and/or one or more other probes, such as detectably labeled probes. In some embodiments, a linear probe is one that comprises a target recognition sequence (e.g., a sequence complementary to a barcode sequence or subunit thereof in the RCA product) and an optically detectable label.

In some embodiments, the detection is spatial, e.g., in two or three dimensions. In some embodiments, the detection is quantitative. In some embodiments, the primary probes, secondary probes, higher order probes, and/or detectably labeled probes may comprise any one of a variety of entities able to hybridize a nucleic acid, e.g., DNA, RNA, LNA, and/or PNA, etc., depending on the application.

In some embodiments, disclosed herein is a multiplexed assay where multiple targets (e.g., nucleic acids such as genes or RNA transcripts, or protein targets) are probed with multiple pluralities of circularizable probes. In some embodiments, detection of barcodes or subsequences of the barcode occurs in a cyclic manner.

In some embodiments, a nucleic acid probe disclosed herein may contain a detectable label such as a fluorophore. In some embodiments, one or more probes of a plurality of nucleic acid probes used in an assay may lack a detectable label, while one or more other probes in the plurality each comprises a detectable label selected from a limited pool of distinct detectable labels (e.g., red, green, yellow, and blue fluorophores), and the absence of detectable label may be used as a separate “color.” As such, detectable labels are not required in all cases. In some embodiments, a primary nucleic acid probe disclosed herein lacks a detectable label. While a detectable label may be incorporated into an amplification product of a probe, such as via incorporation of a modified nucleotide into an RCA product of a circularized probe, the amplification product itself in some embodiments is not detectably labeled. In some embodiments, a probe that binds to the nucleic acid probe or a product thereof comprises a detectable label and may be used to detect the nucleic acid probe or product thereof. In some embodiments, an intermediate probe disclosed herein lacks a detectable label, and a detectably labeled probe that binds to the intermediate probe is used to detect the intermediate probe bound to the amplification product.

In some embodiments, one or more barcode sequences in the circularizable probe bound to the RNA analyte or a complement thereof is detected e.g., by using sequential hybridization of detectably labeled probes, sequencing by hybridization, sequencing by ligation, and/or in situ sequencing e.g., sequencing-by-synthesis (SBS), sequencing-by-avidity (SBA) or sequencing-by-binding (SBB).

In some embodiments, analyzing, e.g., detecting or determining, one or more sequences present in the biological sample is performed using a base-by-base sequencing method, e.g., sequencing-by-synthesis (SBS), sequencing-by-avidity (SBA) or sequencing-by-binding (SBB). In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing is performed by sequencing-by-synthesis (SBS). In some embodiments, a sequencing primer is complementary to sequences at or near the one or more detection sequence or amplification sequences (e.g., barcode(s)). In such embodiments, sequencing-by-synthesis can comprise reverse transcription and/or amplification in order to generate a template sequence from which a primer sequence can bind. In some embodiments, the SBS methods comprise incorporation and/or imaging such as those described in US 2013/0079232; use reagents including, for example, modified and/or labelled nucleotides such as those described in US 2007/0166705 and U.S. Pat. No. 7,057,026; polymerases such as those described in US 2006/0281109, all of which are herein incorporated by reference in their entireties.

In some embodiments, sequencing is performed by sequencing-by-binding (SBB). Various aspects of SBB are described in U.S. Pat. No. 10,655,176 B2 , the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labelling may comprise fluorescence labelling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2 , the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, sequencing is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309:1728-1732, and in U.S. Pat. Nos. 5,599,675; 5,750,341; 6,969,488; 6,172,218; and 6,306,597.

In some embodiments, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences or complements thereof by detecting the corresponding sequences of signal codes detected from sequential hybridization, detection, and removal of sequential pools of intermediate probes and the universal pool of detectably labeled probes. In some cases, the series of signal codes comprise fluorophore sequences assigned to the corresponding barcode sequences or complements thereof. In some cases, the series of signal codes comprise a temporal order of fluorophores assigned to the corresponding barcode sequences or complements thereof. In some embodiments, the detectably labeled probes are fluorescently labeled. In some embodiments, the barcode sequence or complement thereof is performed by sequential probe hybridization as described in US 2021/0340618, the content of which is herein incorporated by reference in its entirety.

In some embodiments, the detecting comprises contacting the biological sample with one or more detectably labeled probes that directly or indirectly hybridize to the barcode sequences or complements thereof (e.g., in amplification products generated using the nucleic acid probes), and dehybridizing the one or more detectably labeled probes. In some embodiments, the contacting and dehybridizing steps are repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte.

In some embodiments, detecting a nucleic acid sequence (e.g., a barcode sequence or barcode subunit) comprises contacting the biological sample with one or more first detectably labeled probes that directly hybridize to the nucleic acid sequence. In some instances, detecting a nucleic acid sequence comprises contacting the biological sample with one or more first detectably labeled probes that indirectly bind to the nucleic acid sequence (e.g., via binding to an intermediate probe that binds to the nucleic acid sequence).

In some embodiments, sequencing is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. In some embodiments, nucleic acid hybridization is used for sequencing. These methods utilize labeled nucleic acid decoder probes that are complementary to at least a portion of a barcode sequence. Multiplex decoding can be performed with pools of many different probes with distinguishable labels.

In some aspects, provided herein are in situ assays using microscopy as a readout, e.g., nucleic acid sequencing, hybridization, or other detection or determination methods involving an optical readout. In some aspects, detection or determination of a sequence of one, two, three, four, five, or more nucleotides of a target nucleic acid is performed in situ in a cell in an intact tissue. In some aspects, the detection or determination is of a sequence associated with or indicative of a target nucleic acid. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product). In some embodiments, a method for spatially profiling analytes such as the transcriptome or a subset thereof in a biological sample is provided. Methods, compositions, kits, devices, and systems for these in situ assays, comprising spatial genomics and transcriptomics assays, are provided. In some embodiments, a provided method is quantitative and preserves the spatial information within a tissue sample without physically isolating cells or using homogenates. In some embodiments, the present disclosure provides methods for high-throughput profiling one or more single nucleotides of interest in a large number of targets in situ, such as transcripts and/or DNA loci, for detecting and/or quantifying nucleic acids in cells, tissues, organs or organisms.

In some aspects, the provided methods comprise imaging the amplification products and/or one or more portions of the polynucleotides, for example, via binding of the detection probe and detecting the detectable label. In some embodiments, the detection probe comprises a detectable label that can be measured and quantitated. A label or detectable label can be a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Background fluorescence can include autofluorescence (that can arise from a variety of sources, including aldehyde fixation, extracellular matrix components, red blood cells, lipofuscin, and the like), as opposed to the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

In some embodiments, one or more fluorescent dyes are used as detectable labels. Commercially available fluorescent dyes include, but are not limited to 4,7-dichlorofluorescein dyes, spectrally resolvable rhodamine dyes, 4,7-dichlororhodamine dyes, cyanine dyes, ether-substituted fluorescein dyes, energy transfer dyes, and xanthine dyes. Labelling can also be carried out with quantum dots. In some embodiments, a fluorescent label comprises a signaling moiety that conveys information through the fluorescent absorption and/or emission properties of one or more molecules. Examples of fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3™-dCTP (cyanine 3-dCTP), Cy3™-dUTP (cyanine 3-dUTP), Cy5™-dCTP (cyanine 5-dCTP), Cy5™-dUTP (cyanine 5 dUTP) (Amersham Biosciences, Piscataway, N.J.), fluorescein-12-dUTP, tetramethylrhodamine-6-dUTP, TEXAS RED®-5-dUTP (red fluorescent dye-dUTP), CASCADE® BLUE-7-dUTP (blue fluorescent dye-dUTP), BODIPY™ FL-14-dUTP (green fluorescent dye-dUTP), BODIPY™ TMR-14-dUTP (orange fluorescent dye-dUTP), BODIPY™ TR-14-dUTP (red fluorescent dye-dUTP), RHODAMINE GREEN™-5-dUTP (green fluorescent dye-dUTP), OREGON GREEN™ 488-5-dUTP (green fluorescent dye-dUTP), TEXAS RED™-12-dUTP (red fluorescent dye-dUTP), BODIPY™ 630/650-14-dUTP (far red fluorescent dye-dUTP), BODIPY™ 650/665-14-dUTP (far red fluorescent dye-dUTP), ALEXA FLUOR™ 488-5-dUTP (green fluorescent dye-dUTP), ALEXA FLUOR™ 532-5-dUTP (yellow fluorescent dye-dUTP), ALEXA FLUOR™ 568-5-dUTP (red/orange fluorescent dye-dUTP), ALEXA FLUOR™ 594-5-dUTP (red fluorescent dye-dUTP), ALEXA FLUORT 546-14-dUTP (orange fluorescent dye-dUTP), fluorescein-12-UTP, tetramethylrhodamine-6-UTP, TEXAS RED™-5-UTP (red fluorescent dye-UTP), mCherry, CASCADE® BLUE-7-UTP (blue fluorescent dye-UTP), BODIPY™ FL-14-UTP (green fluorescent protein-UTP), BODIPY™ TMR-14-UTP (orange fluorescent dye-UTP), BODIPY™ TR-14-UTP (red fluorescent dye-UTP), RHODAMINE GREEN™-5-UTP (green fluorescent dye-UTP), ALEXA FLUOR™ 488-5-UTP (green fluorescent dye-UTP), and ALEXA FLUOR™ 546-14-UTP (orange fluorescent dye-UTP) (Molecular Probes, Inc. Eugene, Oreg.). Methods are known for custom synthesis of nucleotides having other fluorophores.

Other fluorophores available for post-synthetic attachment comprise, but are not limited to, ALEXA FLUOR™ dyes (fluorescent dyes) such as ALEXA FLUOR™ 350 (blue fluorescent dye), ALEXA FLUOR™ 594 (red fluorescent dye), and ALEXA FLUOR™ 647 (far red fluorescent dye); BODIPY™ dyes (fluorescent dyes) such as BODIPY™ FL (green fluorescent dye), BODIPY™ TMR (orange fluorescent dye), and BODIPY™ 650/665 (far red fluorescent dye); Cascade® Blue (blue fluorescent dye), Cascade® Yellow (yellow fluorescent dye), Dansyl, lissamine rhodamine B, Marina Blue™ (blue fluorescent dye), Oregon Green™ 488, Oregon Green™ 514, Pacific Blue, rhodamine 6G, rhodamine green, rhodamine red, tetramethyl rhodamine, Texas Red® (red fluorescent dye) (available from Molecular Probes, Inc., Eugene, Oreg.), Cy2™ (cyanine 2), Cy3.5™ (cyanine 3.5), Cy5.5™ (cyanine 5.5), and Cy7™ (cyanine 7) (Amersham Biosciences, Piscataway, N.J.). FRET tandem fluorophores may also be used, comprising, but not limited to, PerCP-Cy™5.5 (far red fluorescent tandem fluorophore), PE-Cy™5 (red fluorescent tandem fluorophore), PE-Cy™5.5 (red fluorescent tandem fluorophore), PE-Cy™7 (far red fluorescent tandem fluorophore), PE-Texas Red® (red fluorescent tandem fluorophore), APC-Cy™7 (far red fluorescent tandem fluorophore), PE-Alexa™ dyes (e.g., 610, 647, 680), and APC-Alexa™ dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6xHis), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a polynucleotide sequence is indirectly labeled, such as with a hapten that is then bound by a capture agent. Many different hapten-capture agent pairs are available for use. Examples of haptens comprise, but are not limited to, biotin, des-biotin and other derivatives, dinitrophenol, dansyl, fluorescein, cyanine dyes (e.g., Cy5™, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any one of a number of different types of microscopy, e.g., confocal microscopy, two-photon microscopy, light-field microscopy, intact tissue expansion microscopy, and/or CLARITYTM-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity-so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequences are detected in situ, e.g., by incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (i.e., nucleotide sequence) of the incorporated nucleotides or labeled primer extension products can be determined, and consequently, the nucleotide sequence of the corresponding template nucleic acid. Aspects of in situ analysis are described, for example, in Mitra et al., (2003) Anal. Biochem. 320, 55-65, and Lee et al., (2014) Science, 343(6177), 1360-1363; US 2016/0024555; US 2019/0194709; U.S. Pat. Nos. 10,138,509; 10,494,662; 10,179,932.

In some embodiments, sequencing can be performed by sequential fluorescence hybridization (e.g., sequencing by hybridization). Sequential fluorescence hybridization can involve sequential hybridization of detection probes comprising an oligonucleotide and a detectable label.

### V. Sample Preparation

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the biological sample is or comprise a cell pellet or a section of a cell pellet. In some embodiments, the biological sample is or comprise a cell block or a section of a cell block. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample comprises cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein can be any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analyzed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analyzed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample are prepared using formalin-fixation and paraffin-embedding (FFPE). In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes). In some embodiments, the biological sample (e.g., FFPE sample) is permeable after deparaffinization. In some embodiments, processing of the biological sample, such as de-waxing, allows the biological sample to become permeabilized.

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by any suitable methods. In some embodiments, the biological sample is a permeable biological sample. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, is added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ii) Embedding

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some aspects, a biological sample can be embedded in any of a variety of other embedding materials to provide structural substrate to the sample prior to sectioning and other handling steps. In some cases, the embedding material is removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. No. 6,391,937 and materials for sample expansion as described, for example, in U.S. Patent Application Publication Nos. 2017/0253918 and 2018/0052081, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221): 543-548, 2015, the entire contents of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347(6221): 543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

### (iii) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample is segmented using one or more images taken of the stained sample.

In some embodiments, the stain is performed using a lipophilic dye. In some examples, the staining is performed with a lipophilic carbocyanine or aminostyryl dye, or analogs thereof (e. g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples is destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### VI. Compositions, Kits, and Systems

In some embodiments, provided herein are compositions, systems or kits, for example comprising a plurality of circularizable probes comprising a full-length circularizable probe and a less-than-full-length circularizable probe, wherein the plurality of circularizable probes target the same target region of a target nucleic acid, and each circularizable probe of the plurality of circularizable probes comprises a hybridization region comprising a first hybridization sequence at the 5′ end and a second hybridization sequence at the 3′ end, wherein the first hybridization sequence and the second hybridization sequence are complementary to at least a portion of the target region. In some embodiments, at least one probe of the plurality of circularizable probes is different in length than other probes in the plurality. In some embodiments, the compositions, systems or kits comprise a kinase, and a ligase. In some embodiments, the compositions, systems or kits comprise a polymerase. In some embodiments, the compositions, systems or kits comprise a plurality of dNTPs. In some embodiments, disclosed herein is a system that comprises a plurality of amplification products each containing monomeric units of a sequence complementary to a sequence of a circularizable probe. In some embodiments, the plurality of amplification products is formed using the plurality of circularizable probes and any one of the amplification techniques described herein.

Provided herein are systems or kits, for example comprising the plurality of circularizable probes, e.g., full-length circularizable probe and less-than-full-length circularizable probes as described in Section II, and reagents for performing the methods provided herein, for example reagents required for one or more steps comprising hybridization, phosphorylation, ligation, gap fill, amplification, detection, sequencing, and/or sample preparation as described herein. In some embodiments, the system or kit further comprises a target nucleic acid. In some embodiments, the system or kit further comprises a biological sample comprising the target nucleic acid. In some embodiments, any or all of the polynucleotides are DNA molecules. In some embodiments, the target nucleic acid is a messenger RNA molecule. In some embodiments, the system or kit comprises a kinase (e.g., a PNK). In some embodiments, the system or kit comprises a ligase, for instance for forming a ligated circularized probe from the circularizable probe (e.g., padlock probe). In some embodiments, the ligase has DNA-splinted DNA ligase activity. In some embodiments, the system or kit comprises a polymerase, for instance for performing amplification of the circularizable probes. In some embodiments, the polymerase is capable of using the ligated circularized probe as a template for amplification. In some embodiments, the system or kit comprises a primer for amplification.

Provided herein is a system comprising a biological sample comprising a first target nucleic acid molecule and a second target nucleic acid molecule, wherein the biological sample is on a solid support; and a plurality of circularizable probes comprising a full-length circularizable probe and a less-than-full-length circularizable probe, wherein circularizable probes of the plurality of circularizable probes comprises a first hybridization sequence at the 5′ end and a second hybridization sequence at the 3′ end, the full-length circularizable probe and the less-than-full-length circularizable probe comprise phosphorylated 5′ends, the first and second hybridization sequences of the full-length circularizable probe are complementary to a target region of the first target nucleic acid molecule, and the first and second hybridization sequences of the less-than-full-length circularizable probe are complementary to a target region of the second target nucleic acid molecule, and the target region of the first target nucleic acid molecule and the second target nucleic acid molecule are the same target region, wherein the same target region comprises the same nucleic acid sequence.

In some embodiments the plurality of circularizable probes comprises at least one probe different in length than other probes in the plurality of circularizable probes (e.g., a less-than-full-length circularizable probe). For example, the less-than-full-length circularizable probe is at least one nucleotide shorter than a full-length probe in the plurality of circularizable probes, wherein the 5′ terminal nucleotide and the 3′ terminal nucleotide of the full-length probe hybridize to adjacent nucleotides in the target region. In some instances, the less-than-full-length circularizable probe is more than two nucleotides shorter than the full-length probe. In some instances, at least 10% of probes in the plurality of circularizable probes are shorter than the full-length probe. In some instances, the less-than-full-length circularizable probe is missing the at least one nucleotide at the 5′ terminus of the first hybridization sequence compared to the 5′ first hybridization sequence of the full-length probe. In some instances, the plurality of circularizable probes are synthesized from 3′ to 5′ using oligonucleotide synthesis of at least 50 nucleotides, at least 60 nucleotides, at least 70 nucleotides, at least 80 nucleotides, or at least 90 nucleotides. In some embodiments, the system or kit comprises reagents for gap filling the less-than-full-length circularizable probes. For example, the gap of the less-than-full-length circularizable probe is filled using a polymerase and the system or kit comprises a plurality of dNTPs for filling the gap between the first hybridization sequence and the second hybridization sequence.

In some embodiments, the system or kit further comprises reagents for amplifying the plurality of circularized probes using rolling circle amplification (RCA) to generate a plurality of amplification products (e.g., RCA products); and for detecting the RCA products in the biological sample. In some embodiments, the system or kit further comprises a system configured to receive and image the biological sample.

The various components of the system or kit may be present in separate containers or certain compatible components may be pre-combined into a single container. In some embodiments, the kits further contain instructions for using the components of the kit to practice the provided methods.

In some embodiments, the systems or kits comprise reagents and/or consumables required for performing one or more steps of the provided methods. In some embodiments, the systems or kits contain reagents for fixing, embedding, and/or permeabilizing the biological sample. In some embodiments, the systems or kits contain reagents, such as enzymes and buffers for ligation and/or amplification, such as ligases and/or polymerases. In some aspects, the systems or kits also comprise any one of the reagents described herein, e.g., wash buffer and ligation buffer. In some embodiments, the systems or kits contain reagents for detection and/or sequencing, such as barcode detection probes or detectable labels. In some embodiments, the systems or kits optionally contain other components, for example nucleic acid primers, enzymes and reagents, buffers, nucleotides, modified nucleotides, reagents for additional assays.

### VII. Applications

In some aspects, the provided embodiments can be applied in an in situ method of analyzing nucleic acid sequences, such as an in situ transcriptomic analysis, for example from intact tissues or samples in which the spatial information has been preserved. In some aspects, the embodiments are applied in an imaging or detection method for multiplexed nucleic acid analysis. In some aspects, the provided embodiments can be used to identify or detect regions of interest in target nucleic acids.

In some embodiments, the region of interest comprises more than one nucleotide of interest. In some embodiments, the region of interest is a single nucleotide of interest. In some embodiments, the single nucleotide of interest is a single-nucleotide polymorphism (SNP). In some embodiments, the single nucleotide of interest is a single-nucleotide variant (SNV). In some embodiments, the single nucleotide of interest is a single-nucleotide substitution. In some embodiments, the single nucleotide of interest is a point mutation. In some embodiments, the single nucleotide of interest is a single-nucleotide insertion. In some embodiments, the single nucleotide of interest is a single-nucleotide deletion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Gap Fill and Ligation of Synthesized Circularizable Probes

This example demonstrates a workflow involving gap fill and ligation of a plurality of circularizable probes contacted with a biological sample for in situ target nucleic acid detection.

Formalin-fixed, paraffin-embedded (FFPE) samples of a cell block comprising a mixture of Jurkat and Raji cells were cut into thin sections. The sections were placed on slides and dried to dehydrate the sectioned samples. The sections were de-paraffinized using xylene and re-hydrated using an ethanol series (e.g., 96% ethanol followed by 70% ethanol) and nuclease free water before de-crosslinking was performed.

A mixture of circularizable probes (e.g., 189 different padlock probes) designed to hybridize to a panel of target nucleic acids (e.g., mRNAs) was synthesized in two different lots (“Synthesized probes Lot 1” and “Synthesized Probes Lot 2”). A full-length probe of 91 nucleotides in total length was designed to position the 5′ and 3′ the ends of the circularizable probe upon hybridization to the target nucleic acid to allow for ligation. In the full-length probe, the 5′ terminal nucleotide and the 3′ terminal nucleotide hybridize to adjacent nucleotides in the target region of the corresponding target nucleic acid. The circularizable probes of the synthesized lots were assayed for length by performing next generation sequencing on these probes. It was observed the mean percentage of full length probes in a given synthesized lot from two tested synthesis sources was 60.8%-74.2%, indicating a percentage of 25.8%-39.2% probes that were less than full length.

Presence of probes that are less than full length can result in decreased sensitivity in an assay for detecting target nucleic acids.

A third mixture of less-than-full-length circularizable probes targeting the same corresponding target regions was generated (e.g., 96 different padlock probes) except that probes were generated with a single nucleotide missing from the 5′ end, such that each full-length probe was 90 nucleotides in total length (“Synthesized probes Length n−1” where n=full length). The less-than-full-length circularizable probes with the missing nucleotide (“Synthesized probes Length n−1”) were designed to position the 5′ and 3′ the ends upon hybridization to the target nucleic acid to have a gap of 1 nucleotide, such that ligation of the ends is prevented by the gap. In addition, a less-than-full-length circularizable probe missing at least one nucleotide at the 5′ end is also missing a 5′ phosphate modification needed for circularization.

To test the effect of gap filling probes on rescuing less-than-full-length circularizable probes (e.g., such as those with a length of n−1), a workflow performed with gap fill (as depicted in FIG. 2B) and without gap fill was carried out. Mixtures of circularizable probes were incubated with PNK for 30 minutes in vitro to phosphorylate 5′ ends of any less-than-full-length probes prior to being contacted with sectioned samples. As a control, a no PNK treatment condition was provided on all three lots of synthesized probes.

The mixture of circularizable probes (that have either undergone PNK treatment or not) were contacted with the samples on the slides and incubated overnight in hybridization buffer to allow for probe hybridization. After hybridization, the samples were washed and ligation was performed with a ligase for 30 minutes. For gap fill conditions, a gap-fill reaction was performed using a polymerase and incubated for 30 minutes to gap fill (e.g., the one missing nucleotide between the 5′ and 3′ ends of a less-than-full-length probe hybridized to the corresponding target nucleic acid). After gap fill, the sample was contacted with ligase to generate circularized probes. In no gap fill conditions, the samples were not contacted with the polymerase and were not contacted with ligase for a second ligation incubation. Rolling-circle amplification (RCA) of the circularized probes was then performed at approximately 30° C. for 120 minutes. The start of the extension for RCA was synchronized after ligation of the circularizable probes by contacting the sample with a first reaction mixture comprising phi29 polymerase, dNTPs and a dication that is not a catalytic cofactor of the polymerase, thus halting the polymerase. The sectioned samples were incubated with this first reaction mixture at 4° C. (on thermocycler) for 2 hours. The mixture was removed from the sectioned samples and washed. The wash mixture was removed from the sectioned sample and a second reaction mixture comprising dNTPs and Mg2+, a dication that is a catalytic cofactor of the polymerase, was used to initiate RCA.

For detection, fluorescently labeled oligonucleotides were used to hybridize to overhang regions of intermediate probes, and each intermediate probe comprised a hybridization region complementary to a portion of an amplification product (e.g., RCA product) of a circularized probe (e.g., barcodes of the RCA product associated with the target nucleic acid), and after washing, images were obtained to detect the fluorescently labeled oligonucleotides. Multiple cycles of hybridization were performed and images were acquired each cycle using a microscope.

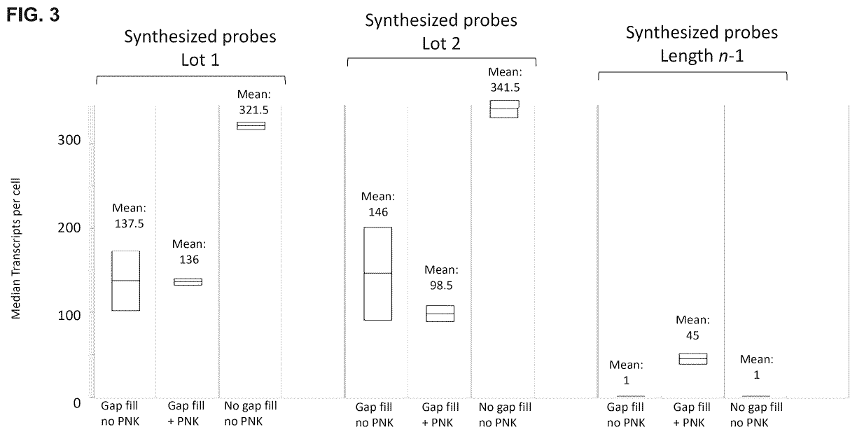

As shown in FIG. 3, in the mixture of circularizable probes generated with a single nucleotide missing from the 5′ end (“Synthesized probes Length n−1”), PNK treatment was needed to rescue the less-than-full-length oligonucleotides and gap filled circularizable probes were successfully ligated to form circularized probes for downstream RCA and detection. As noted, the mixture of circularizable probes generated with a single nucleotide missing from the 5′ end (“Synthesized probes Length n−1”) included half the number of probe targets compared to synthesized probes in Lot 1 and Lot 2, and as such, the median and mean transcripts that were detected were about half.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1-96. (canceled)

97. A method, comprising:
contacting a biological sample with a plurality of circularizable probes comprising a first circularizable probe and a second circularizable probe, wherein:
the plurality of circularizable probes target the same target region of a target nucleic acid, the circularizable probes of the plurality of circularizable probes comprise a first hybridization sequence at a 5′ end and a second hybridization sequence at a 3′ end, and
the second circularizable probe is missing at least one nucleotide at the 5′ end of the first hybridization sequence corresponding to the nucleotide at the 5′ terminus of the first circularizable probe,
wherein the first hybridization sequence and the second hybridization sequence hybridize to a sequence within the target region, and
wherein the 5′ terminal nucleotide and the 3′ terminal nucleotide of the first circularizable probe hybridize to adjacent nucleotides in the target region;
filling a gap between the first hybridization sequence and the second hybridization sequence of the second circularizable probe to form a gap-filled circularizable probe; and
forming a plurality of circularized probes from the first circularizable probe and the gap-filled circularizable probe in the biological sample.

98. The method of claim 97, wherein at least 20% of probes in the plurality of circularizable probes are second circularizable probes.

99. The method of claim 97, wherein the plurality of circularizable probes are synthesized with a coupling reaction that proceeds from 3′ to 5′ and the synthesis generates a plurality of second circularizable probes comprising a truncated sequence from its intended full length.

100. The method of claim 99, wherein synthesis of the plurality of circularizable probes comprises coupling of at least 70 nucleotides.

101. The method of claim 97, comprising contacting the biological sample with a ligase during or prior to filling the gap between the first hybridization sequence and the second hybridization sequence.

102. The method of claim 101, further comprising contacting the biological sample with an additional ligase after filling the gap between the first hybridization sequence and the second hybridization sequence to form the plurality of circularized probes.

103. The method of claim 97, wherein forming the plurality of circularized probes comprises performing an RNA templated ligation.

104. The method of claim 97 wherein the second circularizable probe comprises a non-phosphorylated 5′ end.

105. The method of claim 104, further comprising using a kinase to phosphorylate the non-phosphorylated 5′ end of the second circularizable probe.

106. The method of claim 105, wherein the phosphorylation is performed in the biological sample after contacting the biological sample with the plurality of circularizable probes.

107. The method of claim 105, wherein the phosphorylation of the non-phosphorylated 5′ end of the second circularizable probe is performed in a ligation buffer.

108. The method of claim 97, further comprising generating a plurality of amplification products using the plurality of circularized probes as templates.

109. The method of claim 108, wherein the plurality of amplification products comprises a plurality of rolling circle amplification (RCA) products.

110. The method of claim 97, wherein circularized probes of the plurality of circularized probes comprise one or more barcode sequences associated with the target nucleic acid.

111. The method of claim 110, further comprising detecting the one or more barcode sequences or complementary sequences thereof in the biological sample.

112. The method of claim 111, wherein detecting the one or more barcode sequences or complement thereof comprises:
contacting the biological sample with a detectably labeled probe, wherein the detectably labeled probe comprises a hybridization region complementary to one barcode sequence or complement thereof of the one or more barcode sequences or complements thereof; and
detecting complexes formed between the one or more barcode sequences or complements thereof and the detectably labeled probe.

113. The method of claim 111, wherein detecting the one or more barcode sequences or complements thereof comprises:
contacting the biological sample with a universal pool of detectably labeled probes and a first pool of intermediate probes, wherein an intermediate probe of the first pool of intermediate probes comprises hybridization regions complementary to the one or more barcode sequences or complements thereof and reporter regions complementary to a detectably labeled probe of the universal pool of detectably labeled probes; and
detecting complexes formed between the barcode sequences or complements thereof, the intermediate probe of the first pool of intermediate probes, and the detectably labeled probe of the universal pool of detectably labeled probes.

114. The method of claim 111, wherein detecting the one or more barcode sequences or complements thereof comprises performing in situ sequencing-by-synthesis (SBS), sequencing-by-avidity (SBA) or sequencing-by-binding (SBB) in the biological sample.

115. The method of claim 97, wherein the target nucleic acid is an mRNA.

116. The method of claim 97, wherein the biological sample is a cell or tissue sample.
